(19) United States
(12) Patent Application Publication
(10) Pub. No.: US 2024/0091357 A1
(43) Pub. Date: Mar. 21, 2024

(54) ENGINEERED IMMUNE CELL

(71) Applicant: AUTOLUS LIMIRED, London (GB)

(72) Inventors: Martin Pulé, London (GB); Shaun Cordoba, London (GB); Shimobi Onuoha, London (GB); Simon Thomas, London (GB); Thomas Grothier, London (GB)

(21) Appl. No.: 17/767,806

(22) PCT Filed: Oct. 9, 2020
(86) PCT No.: PCT/GB2020/052514

§ 371 Date: Apr. 8, 2022

(30) Foreign Application Priority Data
Oct. 9, 2019 (GB) 1914611.7

(51) Int. Cl. A61K 39/00; C07K 14/705; C07K 14/725; C07K 14/74; C07K 16/28; C07K 16/40; C12N 5/0783

(52) U.S. Cl. CPC A61K 39/4632; A61K 39/4611; C07K 14/7051; C07K 14/70517; C07K 14/70539; C07K 16/2809; C07K 16/2818; C07K 16/2833; C07K 16/40; C12N 5/0636; A61K 2239/13; A61K 2239/22; A61K 2239/24; A61K 2239/26; C07K 2317/569; C07K 2319/02; C07K 2319/03; C07K 2319/04; C12N 2510/00

(57) ABSTRACT

The present invention relates to an engineered immune cell which comprises: (i) a target binding polypeptide comprising a target-binding domain and a first protein interaction domain, and (ii) a localising polypeptide comprising a second protein interaction domain, which binds to the first protein binding domain, and an intracellular retention signal. When the target binding polypeptide binds its target protein and also the localising polypeptide, expression of the target protein at the cell surface is reduced or eliminated because the target protein is retained in an intracellular compartment.

### FIELD OF THE INVENTION

The present invention relates to approaches for controlling the expression of target proteins in an engineered immune cell. In particular, the invention relates to approaches for controlling the expression of at least two target proteins via retention in an intracellular compartment.

### BACKGROUND TO THE INVENTION

Classically, surface-expressed proteins are directed to the endoplasmic reticulum (ER) by signal peptide sequences and are anchored in the membrane of the ER by hydrophobic helical transmembrane domain or domains. These proteins fold in the ER and migrate through the secretory pathway to the cell surface.

Some proteins within the ER are directed to the Golgi apparatus rather than the secretory pathway and hence are not expressed on the cell surface. Several motifs direct proteins to the Golgi apparatus.

One of the most characterized such motif is the “SEKDEL” motif. Expression of this motif on the extreme carboxy-terminus of a protein will direct it from the ER to the Golgi apparatus. This motif can be exploited to direct proteins, which do not normally migrate there, to the Golgi from the ER. For instance, Pelham et al. (Methods Enzymol. 327, 279-283 (2000)) described that expression of a single-chain variable fragment (scFv) with a carboxy-terminal sekdel can direct its cognate target to the Golgi and hence reduce or knock-out surface expression of the cognate target (see FIG. 1).

Due to their inherent stability and simplicity single-domain antibody fragments (dAbs) are well suited to “SEKDEL knockdown”.

In some instances, reduction of surface expression of multiple proteins may be desired. This requires co-expression of many different dAb-sekdel fusions. Co-expression of multiple transgenic proteins is difficult and typically requires multiple transductions (which carries risk of insertional mutagenesis) and/or multiple internal promoters (which results in promoter interference and silencing). Further, motifs such as internal ribosome entry sequences (IRES) result in much lower expression of down-stream protein(s).

One alternative is the use of the foot-and-mouth disease 2A or 2A-like sequences. Donnelly et al. (J. Gen. Virol. 82, 1027-1041 (2001)) described these short peptide sequences which are cleaved very efficiently. For some settings, a single open-reading frame with 2A peptides is the only way to express multiple transgenic proteins and hence, in some settings to date, the use of a 2A peptide is the only way multiple surface proteins can be knocked-out with an SEKDEL knockdown.

A limitation of the 2A peptide is its cleavage leads to the retention of 15-20 residual bases of the 2A peptide (except for the final proline) at the carboxy terminus of the transgenic protein. These residual bases may inhibit sekdel recognition—for example by masking the SEKDEL motif when positioned immediately C-terminal to SEKDEL intracellular retention signal.

Accordingly, there remains a need for approaches which enable the reduction or knock-down of multiple target proteins.

### SUMMARY OF THE INVENTION

The present inventors have developed a range of engineered proteins which are capable of reducing or knocking-down the expression of one or more target protein(s).

In a first aspect, the invention provides an engineered immune cell which comprises:
(i) a target-binding polypeptide comprising a target-binding domain and a first protein interaction domain, and
(ii) a localising polypeptide comprising a second protein interaction domain, which binds to the first protein binding domain, and an intracellular retention signal.

The cell may comprise:
(i) at least two target-binding polypeptides, each of which comprise a target-binding domain and a first protein interaction domain, and
(ii) a localising polypeptide comprising a second protein interaction domain, which binds to the first protein binding domain of each of the target binding polypeptides, and an intracellular retention signal.

The at least two target-binding polypeptides may bind to the same target protein. For example, the at least two target-binding polypeptides may bind to different epitopes of the same target protein. In this respect, the two or more target-binding polypeptides may act cooperatively to cause retention of the target protein in an intracellular compartment.

Alternatively, the at least two target-binding polypeptides may bind to different target proteins. In this respect, the two or more target-binding polypeptides may act independently to cause retention of two or more target proteins in an intracellular compartment.

The intracellular retention signal may be selected from the following group: a Golgi retention sequence; a trans-Golgi network (TGN) recycling signal; an endoplasmic reticulum (ER) retention sequence; a proteasome localization sequence or a lysosomal sorting signal.

For example, the intracellular retention signal may be selected from:
a) a Golgi retention sequence which comprises an amino acid sequence selected from: SEKDEL(SEQ ID NO: 1), KDEL (SEQ ID NO: 2), KKXX (SEQ ID NO: 3), KXKXX (SEQ ID NO: 4), a tail of adenoviral E19 protein comprising the sequence KYKSRRSFIDEKKMP (SEQ ID NO: 5), a fragment of HLA invariant chain comprising the sequence MHRRRSRSCR (SEQ ID NO: 6), KXD/E (SEQ ID NO: 7) or a YQRL (SEQ ID NO: 8), wherein X is any amino acid; and/or
b) an endoplasmic reticulum retention domain selected from: Ribophorin I, Ribophorin II, SEC61 or cytochrome b5; and/or
c) an intracellular retention signal comprising any sequence shown in Tables 1 to 5.

The or each target-binding domain may comprise a single domain antibody (sdAb).

The target protein may, for example, be a component of a CD3/T-cell receptor (TCR) complex, a cytokine, a human leukocyte antigen (HLA) class I molecule, an MHC class II molecule, a receptor that downregulates immune response, a ligand expressed on T cells, or a cytosolic protein that modulates the immune response.

The target protein may be selected from the following group:
(i) a component in a CD3/TCR complex selected from: CD3ε, TCRα, TCRαβ, TCRγ, TCRδ, CD3δ, CD3γ, and CD3ζ;
(ii) an HLA Class I molecule selected from: B2-microglobulin, α1-microglobulin, α2-microglobulin, and α3-microglobulin;
(iii) an MHC class II molecule selected from: HLA-DP, HLA-DM, HLA-DOA, HLA-DOB, HLA-DQ and HLA-DR;
(iv) a receptor that downregulates immune response selected from: programmed cell death protein 1 (PD-1), cytotoxic T-lymphocyte-associated protein 4 (CTLA-4), T-cell immunoglobulin and mucindomain containing-3 (Tim3), killer immunoglobulin-like receptors (KIRs), CD94, NKG2A, TIGIT, BTLA, Fas, TBR2, LAG3 and a protein tyrosine phosphatase;
(v) a ligand expressed on T cells selected from: CD5, CD7 and CD2
(vi) a cytosolic protein which modulates the immune response selected from: Csk, SHP1, SHP2, Zap-70, SLP76 and AKT.

The cell may further comprises a chimeric antigen receptor (CAR) or a transgenic T cell receptor (TCR).

In a second aspect, the present invention provides a nucleic acid construct which comprises:
(i) a first nucleic acid sequence which encodes a target binding polypeptide comprising a target-binding domain and a first protein interaction domain, and
(ii) a second nucleic acid sequence which encodes a localising polypeptide comprising a second protein interaction domain, which binds to the first protein binding domain, and an intracellular retention signal.

The nucleic acid construct may have the following general structure:

A-coexpr-C
in which:
“A” is a nucleic acid sequence encoding the target-binding polypeptide
“coexpr” is a sequence enabling the coexpression of the target polypeptide and localising polypeptide as separate entities; and
“C” is a nucleic acid sequence encoding the localising polypeptide

The nucleic acid construct may comprise:
(i) a plurality of nucleic acid sequences each of which encodes a target binding polypeptide comprising a target-binding domain and a first protein interaction domain, and
(ii) a nucleic acid sequence which encodes a localising polypeptide comprising a second protein interaction domain, which binds to the first protein binding domain, and an intracellular retention signal.

The nucleic acid construct may have the following general structure:

A-coexpr1-B-coexpr2-C

in which:
“A” is a nucleic acid sequence encoding a first target-binding polypeptide;
“B” is a nucleic acid sequence encoding a second target-binding polypeptide
“coexpr1” and “coexpr2” which may be the same or different, are sequences enabling the coexpression of the three polypeptides as separate entities; and
“C” is a nucleic acid sequence encoding the localising polypeptide.

Where the nucleic acid construct encodes three target-binding polypeptides, it may have the following general structure:

A-coexpr1-B-coexpr2-D-coexpr3-C
in which:
“A” is a nucleic acid sequence encoding a first target-binding polypeptide;
“B” is a nucleic acid sequence encoding a second target-binding polypeptide;
“D” is a nucleic acid sequence encoding a third target-binding polypeptide;
“coexpr1”, “coexpr2” and “coexpr3” which may be the same or different, are sequences enabling the coexpression of the four polypeptides as separate entities; and
“C” is a nucleic acid sequence encoding the localising polypeptide.

In all nucleic acid constructs, the nucleic acid sequence encoding the localising peptide may be located such that it is expressed at the C-terminus. In this way, the intracellular retention signal is unaffected by any residues left following cleavage at the coexpression sequence (for example where the co-expression sequence encodes a self-cleaving peptide, such as the 2A peptide).

In a third aspect, the invention provides a kit of nucleic acid sequences which comprises:
(i) a first nucleic acid sequence which encodes a target binding polypeptide comprising a target-binding domain and a first protein interaction domain, and
(ii) a second nucleic acid sequence which encodes a localising polypeptide comprising a second protein interaction domain, which binds to the first protein binding domain, and an intracellular retention signal.

The kit of nucleic acid sequences may comprise:
(i) a plurality of nucleic acid sequences each of which encodes a target binding polypeptide comprising a target-binding domain and a first protein interaction domain, and
(ii) a nucleic acid sequence which encodes a localising polypeptide comprising a second protein interaction domain, which binds to the first protein binding domain, and an intracellular retention signal.

In a fourth aspect, the invention provides a vector which comprises a nucleic acid construct according to the second aspect of the invention.

In a fifth aspect, the invention provides a kit of vectors which comprises:
(i) a first vector comprising a nucleic acid sequence which encodes a target binding polypeptide comprising a target-binding domain and a first protein interaction domain, and
(ii) a second vector comprising a nucleic acid sequence which encodes a localising polypeptide comprising a second protein interaction domain, which binds to the first protein binding domain, and an intracellular retention signal.

The kit of vectors may comprise:
(i) a plurality of vectors each of which comprises a nucleic acid sequence encoding a target binding polypeptide comprising a target-binding domain and a first protein interaction domain, and
(ii) a vector comprising a nucleic acid sequence which encodes a localising polypeptide comprising a second protein interaction domain, which binds to the first protein binding domain, and an intracellular retention signal.

In a sixth aspect, the invention provides a method for making an engineered immune cell according to the first aspect of the invention, which comprises the step of introducing into an immune cell a nucleic acid construct according to the second aspect of the invention; a kit of nucleic acid sequences according to the third aspect of the invention; a vector according to the fourth aspect of the invention; or a kit of vectors according to the fifth aspect of the invention, into a cell ex vivo.

In a seventh aspect, the invention provides a pharmaceutical composition which comprises a plurality of engineered immune cells according to the first aspect of the invention.

In an eighth aspect pharmaceutical composition according to the seventh aspect of the invention for use in treating and/or preventing a disease.

In a ninth aspect, there is provided a method for treating a disease, which comprises the step of administering a pharmaceutical composition according to the seventh aspect of the invention to a subject in need thereof.

In a tenth aspect, there is provided the use of a cell according to the first aspect of the invention in the manufacture of a medicament for the treatment of a disease.

The disease may be cancer.

### FURTHER ASPECTS OF THE INVENTION

Further aspect of the invention are summarised in the following numbered paragraphs:
1. An engineered immune cell comprising one or more nucleic acid constructs which together encode at least two target-binding domains, wherein the one or more nucleic acid constructs together contain a single nucleotide sequence encoding an intracellular retention signal which, when co-expressed in the cell, controls the cellular localisation of each of the target-binding domains.
2. An engineered immune cell according to paragraph 1, wherein the one or more nucleic acid construct(s) encodes at least one engineered protein which comprises the at least two target-binding domains coupled to the intracellular retention signal.
3. An engineered immune cell according to paragraph 1 or 2, wherein the at least two target-binding domains are coupled to the intracellular retention signal via linkers, preferably peptide linkers.
4. An engineered immune cell according to paragraph 1 to 3, wherein said at least two target-binding domains are coupled to the intracellular retention signal by at least one heteromultimeric protein.
5. An engineered immune cell according to paragraph 4, wherein said at least one heteromultimeric protein comprises at least two polypeptides coupled by a disulphide bond(s).
6. An engineered immune cell according to any preceding paragraph, wherein said engineered protein comprises a first peptide subunit comprising a first target-binding domain and an intracellular retention signal and a second peptide subunit comprising at least a second target-binding domain; wherein the first and second peptide subunits are coupled, preferably by a peptide linker or one or more disulphide bonds.
7. An engineered immune cell according to paragraph 1, wherein each of the at least two target-binding domains and the intracellular retention signal are encoded as separate polypeptides; each of the polypeptides comprising the target-binding domains further comprises a first protein interaction domain and the polypeptide comprising the intracellular retention signal further comprises a second protein interaction domain wherein the first and second protein interaction domains are capable of binding to each other.
8. An engineered immune cell according to any preceding paragraph, wherein said engineered protein comprises at least three target-binding domains.
9. An engineered immune cell according to paragraph 8, wherein one polypeptide chain comprises at least two target-binding domains and one polypeptide chain comprises at least one target-binding domain and an intracellular retention signal.
10. An engineered immune cell according to any preceding paragraph, wherein the intracellular retention signal directs the protein to a Golgi, endosomal or lysosomal compartment.
11. An engineered immune cell according to any preceding paragraph, wherein the intracellular retention signal is selected from the following group: a Golgi retention sequence; a trans-Golgi network (TGN) recycling signal; an endoplasmic reticulum (ER) retention sequence; a proteasome localization sequence or a lysosomal sorting signal.
12. An engineered immune cell according to any preceding paragraph wherein:
   a) the Golgi retention sequence comprises an amino acid sequence selected from: SEKDEL(SEQ ID NO: 1), KDEL (SEQ ID NO: 2), KKXX (SEQ ID NO: 3), KXKXX (SEQ ID NO: 4), a tail of adenoviral E19 protein comprising the sequence KYKSRRSFIDEKKMP (SEQ ID NO: 5), a fragment of HLA invariant chain comprising the sequence MHRRRSRSCR (SEQ ID NO: 6), KXD/E (SEQ ID NO: 7) or a YQRL (SEQ ID NO: 8), wherein X is any amino acid; and/or
   b) the endoplasmic reticulum retention domain is selected from: Ribophorin I, Ribophorin II, SEC61 or cytochrome b5; and/or
   c) an intracellular retention signal comprising any sequence shown in Tables 1 to 5.
13. An engineered immune cell according to any preceding paragraph, wherein at least one target-binding domain comprises an antibody, an antibody fragment or antigen binding fragment, a single-chain variable fragment (scFv), a domain antibody (dAb), a single domain antibody (sdAb), a VHH/nanobody, a nanobody, an affibody, a fibronectin artificial antibody scaffold, an anticalin, an affilin, a DARPin, a VNAR, an Body, an affimer, a fynomer, an abdurin/nanoantibody, a centyrin, an alphabody or a nanofitin.
14. An engineered immune cell according to any preceding paragraph, wherein at least one target-binding domain is a domain antibody (dAb) or a single-chain variable fragment (scFv).
15. An engineered immune cell according to any preceding paragraph, wherein at least one target is selected from: a cytosolic protein, an intracellular protein, an extracellular protein, and a transmembrane protein.
16. An engineered immune cell according to any preceding paragraph, wherein at least two targets are localised in different cellular compartments.
17. An engineered immune cell according to paragraph 16, wherein said at least one engineered protein comprises at least one transmembrane domain.
18. An engineered immune cell according to paragraph 17, wherein:
   at least one target is an extracellular protein and at least one target is an intracellular protein; or
   at least one target is a cytosolic protein and at least one target is an endoplasmic reticulum lumen protein.
19. An engineered immune cell according to any preceding paragraph wherein at least one target-binding domain binds to a component of a CD3/T-cell receptor (TCR) complex, a cytokine, a human leukocyte antigen (HLA) class I molecule, a receptor that downregulates immune response, a ligand expressed on T cells, or a cytosolic proteins that modulate the immune response.
20. An engineered immune cell according to paragraph 19, wherein the component in a CD3/TCR complex is CD3ε, TCRα, TCRαβ, TCRγ, TCRδ, CD3δ, CD3γ, or CD3ζ.
21. The engineered immune cell according to paragraph 19, wherein the HLA Class I molecule is B2-microglobulin, α1-microglobulin, α2-microglobulin, or α3-microglobulin.
22. The engineered immune cell of paragraph 19, wherein the receptor that downregulates immune response is selected from programmed cell death protein 1 (PD-1), cytotoxic T-lymphocyte-associated protein 4 (CTLA-4), T-cell immunoglobulin and mucindomain containing-3 (Tim3), killer immunoglobulin-like receptors (KIRs), CD94, NKG2A, TIGIT, BTLA, Fas, TBR2, LAG3 or a protein tyrosine phosphatase.
23. The engineered immune cell of paragraph 19, wherein the cytosolic protein which modulates the immune response is selected from Csk, SHP1, SHP2, Zap-70, SLP76 and AKT.
24. The engineered immune cell of paragraph 19, wherein the ligand expressed on T cells is CD5, CD7 or CD2.
25. An engineered immune cell according to any preceding paragraph, wherein the cell is a T cell, an alpha-beta T cell, a NK cell, a gamma-delta T cell, or a cytokine induced killer cell.
26. An engineered immune cell according to any preceding paragraph, wherein the cell further comprises a chimeric antigen receptor (CAR) or transgenic T cell receptor (TCR).
27. An engineered immune cell according to any preceding paragraph, wherein the cell further comprises at least one marker, preferably said marker is an extracellular binding domain comprising at least one mAb-specific epitope.
28. A nucleic acid construct which comprises the following structure:

A-X-B-C
in which:
A and B are nucleic acid sequences encoding a target-binding domain as defined in any one of paragraphs 1 to 27; X is a linker as defined in any one of paragraphs 1 to 27; and C is an intracellular retention signal as defined in any one of paragraphs 1 to 27.
29. A nucleic acid construct according to paragraph 28, further comprising one or more additional nucleic acid sequences encoding an additional target-binding domain(s) as defined in any one of paragraphs 1 to 27.
30. A nucleic acid construct according to paragraph 28, further comprising a nucleic acid sequence which encodes a CAR or transgenic TCR.
31. A nucleic acid construct according to any one of paragraphs 28-30, further comprising a nucleic acid sequence encoding at least one marker, preferably wherein said marker is an extracellular binding domain comprising at least one mAb-specific epitope.
32. A nucleic acid construct according to paragraph 30 or 31, wherein the nucleic acid sequence encoding said CAR, transgenic TCR or marker is adjacent to a nucleic acid sequence encoding a self cleaving peptide.
33. A nucleic acid construct according to paragraph 32, wherein the self-cleaving peptide is a 2A self cleaving peptide from an aphtho- or a cardiovirus or a 2A-like peptide.
34. A nucleic acid construct according to paragraph 32 or 33, wherein a nucleic acid sequence encoding a 2A self-cleaving peptide is adjacent to the nucleic acid sequence encoding said CAR, transgenic TCR or marker on the 3′ end.
35. A kit of nucleic acid sequences comprising:
   (i) a nucleic acid sequence encoding a protein which comprises at least one target-binding domain linked to an intracellular retention signal; and
   (ii) a nucleic acid sequence encoding a protein which is capable of coupling to the protein encoded by (i) and which comprises at least one target-binding domain.
36. The kit of nucleic acid sequences according to paragraph 35, wherein the nucleic acid sequence as defined in (i), the nucleic acid sequence as defined in (ii) or an additional nucleic acid(s) encode one or more of: a CAR or transgenic TCR and/or a marker, preferably said marker is an extracellular binding domain comprising at least one mAb-specific epitope.
37. A vector which comprises a nucleic acid construct according to any one of paragraphs 28 to 34.
38. A method for making an engineered immune cell according to any of paragraphs 1 to 27, which comprises the step of introducing into an immune cell a nucleic acid construct according to any one of paragraphs 28 to 34, a group of nucleic acid sequences as defined in paragraph 35 or 36, or a vector according to paragraph 37.
39. A method for controlling the cellular localisation of at least two target proteins, which comprises the steps of: introducing to a cell a nucleic acid construct according to any one of paragraph 28 to 34, a group of nucleic acid sequences as defined in paragraph 35 or 36; or a vector according to paragraph 37.
40. A method according to paragraph 39, wherein the expression of one or more target proteins at the cell surface is reduced or eliminated and/or wherein the target protein is retained in an intracellular compartment.
41. A pharmaceutical composition which comprises an engineered immune cell according to any one of paragraphs 1 to 27, a nucleic acid construct according to any one of paragraphs 28 to 34, a group of nucleic acid sequences as defined in paragraph 35 or 36, or a vector according to paragraph 37.
42. A pharmaceutical composition which comprises a cell according to any of paragraphs 1 to 27 or a cell obtainable by a method according to any of paragraphs 38 to 40.
43. A pharmaceutical composition according to paragraph 41 or 42 for use in treating and/or preventing a disease.
44. A method for treating and/or preventing a disease, which comprises the step of administering a pharmaceutical composition according to paragraph 41 or 42 to a subject in need thereof.
45. A method according to paragraph 44, which comprises the following steps:
   (i) isolation of a cell containing sample;
   (ii) introduction of the nucleic acid construct according to any one of paragraphs 28 to 34, a group of nucleic acid sequences as defined in paragraph 35 or 36, or a vector according to paragraph 37; and
   (iii) administering the cells from (ii) to a subject.
46. The method according to paragraph 45, wherein the nucleic acid construct or vector is introduced by transduction or transfection.
47. The method according to paragraph 44 to 46, wherein the cell is autologous.
48. The method according to paragraph 44 to 46, wherein the cell is allogenic.
49. The use of a pharmaceutical composition according to paragraph 41 to 43 in the manufacture of a medicament for the treatment and/or prevention of a disease.

The present inventors have thus developed a range of engineered proteins which are capable of reducing or knocking-down the expression of one or more target protein(s). The present engineered proteins are based on architectures which enable multiple target proteins to be directed to a desired intracellular compartment by coupling at least two target-binding domains to a single intracellular retention signal.

As used herein, the term “which together encode” is used to mean that the recited entities are encoded by nucleic acid sequences provided by the one or more nucleic acid constructs when taken as a whole. In other words, the at least two target-binding domains and single nucleotide sequence encoding an intracellular retention signal are encoded between the nucleic acid sequences present in the one or more nucleic acid constructs. For example, the at least two target-binding domains and single nucleotide sequence encoding an intracellular retention signal may be provided on a single nucleic acid construct. Alternatively, a first target-binding domain and a nucleotide sequence encoding an intracellular retention signal may be provided on a first nucleic construct and a second target-binding domain may be provided on a second nucleic acid construct. As will be apparent, multiple variations and arrangements which fall within the present invention may be envisaged, providing that between the one or more nucleic acid constructs at least two target-binding domains and single nucleotide sequence encoding an intracellular retention signal are encoded.

The term “single nucleotide sequence encoding an intracellular retention signal” means that the one or more nucleic acid constructs only encode one intracellular retention signal which controls the cellular localisation of each of the target-binding domains. Accordingly, the present engineered proteins are based on architectures which enable multiple target proteins to be directed to a desired intracellular compartment by coupling at least two target-binding domains to a single intracellular retention signal provided by the one or more nucleic acid construct(s).

“When co-expressed in a cell” is used herein to mean that the amino acid sequence providing the at least two target-binding domains and the amino acid sequence providing the intracellular retention signal are expressed at the same time in the cell of the invention. The relevant amino acid sequences may be present as part of one or multiple polypeptides, as defined herein.

The term “controls the cellular localisation of each of the target-binding domains” means that the intracellular retention signal directs or maintains the protein in which it is encompassed to a cellular compartment other than that to which it would be directed in the absence of the intracellular retention signal. Suitably, the intracellular retention signal directs or maintains the protein in which it is encompassed to a cellular compartment other than the cell surface membrane or to the exterior of the cell.

Without wishing to be bound by theory, the engineered proteins of the present invention have utility in a variety of potential settings. By way of example, they may facilitate the generation of analogous CAR T cells by targeting proteins such as MHC class I, β₂ microglobulin, MHC class II and/or TCR for knock-down in order to reduce or prevent graft-vs-host or host-vs-graft disease. They may also be used to reduce suppression of CAR T cells and increase sensitivity through the knock-down of inhibitory protein such as: surface proteins PD1, TIGIT, BTLA, TIM3, Fas, CTLA, TBR2 and LAG3 and cytosolic proteins SHP1, SHP2 and CSK. Further, the present engineered proteins may reduce fratricide when targeting a group of ligands also expressed on the CAR T cells such as CD5, CD7 and CD2.

### BRIEF DESCRIPTION OF THE FIGURES

FIG. 1: A) Illustrative traffic pathway of a surface expressed protein to the cell membrane via the endoplasmic reticulum and the golgi. B) Illustrative retention of CD36 in intracellular compartments by a dAb comprising a KDEL motif.

FIG. 2: Illustrative embodiment of a single polypeptide chain comprising multiple target-binding domains linked to a single KDEL motif.

FIG. 3: A) Illustrative embodiment of two polypeptide chains consisting of multiple target-binding domains linked to a KDEL motif encoded on a single construct. B) Illustrative embodiment of two polypeptide chains consisting of multiple target-binding domains linked to a KDEL motif encoded on two separate constructs. C) Illustrative embodiment of three polypeptide chains consisting of multiple target-binding domains linked to a KDEL motif FIG. 4: Illustrative embodiment of several polypeptide chains each comprising at least one target-binding domain and a tag followed by a final polypeptide chain consisting of a tag-binding protein followed by KDEL motif.

FIG. 5: Illustrative embodiment of several polypeptide chains each comprising at least one target-binding domain and a tag followed by a final polypeptide chain comprising at least two transmembrane domains, a lumen residing tag-binding protein, a cytosolic residing tag-binding protein and a C-terminus KDEL residing in the lumen.

FIG. 6: KDEL driven TCR knock-down can be mediated through a dual polypeptide chain construct A) PBMC's were transduced to express either a single polypeptide encoding anti-TCR_VHH directly linked to a KDEL sequence; or two polypeptide chains: with the first encoding an anti-TCR_VHH linked to an ALFA_peptide and the second encoding an anti-ALFA_peptide_VHH directly linked to a KDEL sequence. The two polypeptides were separated by a self-cleaving 2A peptide. As a negative control the aTCR_VHH was substituted with an irrelevant VHH binder. All constructs contained an IRES-eBFP marker for transduction. B) after 4 days of transduction, PBMCs were stained for surface CD3. Results are from four independent donors.

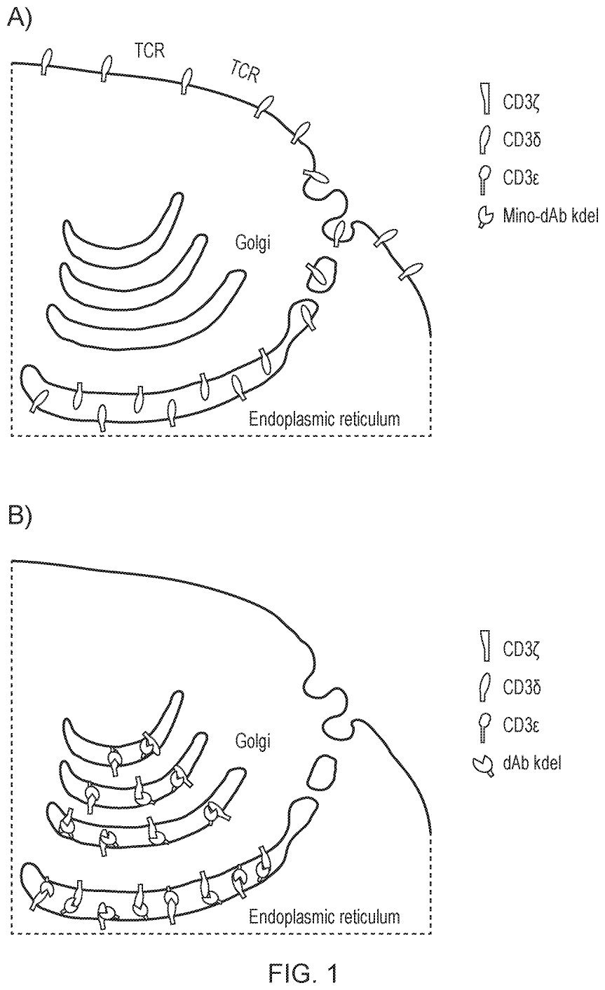

### DETAILED DESCRIPTION OF THE INVENTION

The present invention relates to an engineered immune cell comprising one or more nucleic acid constructs which together encode at least two target-binding domains, wherein the one or more nucleic acid constructs together contain a single nucleotide sequence encoding an intracellular retention signal which, when co-expressed in the cell, controls the cellular localisation of each of the target-binding domains. The present invention extends to an engineered immune cell comprising at least one engineered protein which comprises at least two target-binding domains coupled to an intracellular retention signal. The engineered protein is capable of controlling the cellular localisation of at least two proteins. The invention also relates to nucleic acid constructs, kits of nucleic acid sequences and vectors encoding at least one engineered protein which comprises at least two target-binding domains coupled to an intracellular retention signal. The invention also extends to pharmaceutical compositions comprising cells, nucleic acid constructs or vectors according to the invention and the use of said pharmaceutical compositions for the treatment or prevention of disease.

Engineered Immune Cell

The present invention relates to an engineered immune cell comprising one or more nucleic acid constructs which together encode at least two target-binding domains, wherein the one or more nucleic acid constructs together contain a single nucleotide sequence encoding an intracellular retention signal which, when co-expressed in the cell, controls the cellular localisation of each of the target-binding domains.

An “engineered immune cell” as used herein means an immune cell which has been modified to comprise or express a nucleic acid sequence which is not naturally encoded by the cell. Methods for engineering cells are known in the art and include but are not limited to genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid sequence into a cell.

Suitably, an engineered cell is a cell that has been modified, or whose genome has been modified, e.g. by transduction or by transfection. Suitably, an engineered cell is a cell that has been modified, or whose genome has been modified, by retroviral transduction. Suitably, an engineered cell is a cell that has been modified, or whose genome has been modified, by lentiviral transduction.

In one aspect, the engineered immune cell is an engineered cytolytic immune cell.

“Cytolytic immune cell” as used herein is a cell which directly kills other cells. Cytolytic cells may kill cancerous cells; virally infected cells or other damaged cells. Cytolytic immune cells include T cells and Natural killer (NK) cells.

Cytolytic immune cells can be T cells or T lymphocytes which are a type of lymphocyte that play a central role in cell-mediated immunity. T cells can be distinguished from other lymphocytes, such as B cells and NK cells, by the presence of a TCR on their cell surface.

Cytolytic T cells (TC cells or CTLs) destroy virally infected cells and tumour cells, and are also implicated in transplant rejection. CTLs express the CD8 at their surface. CTLs may be known as CD8+ T cells. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Suitably, the cell of the present invention may be a T-cell. Suitably, the T cell may be an alpha-beta T cell. Suitably, the T cell may be a gamma-delta T cell.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

Suitably, the cell of the present invention may be a wild-type killer (NK) cell. Suitably, the cell of the present invention may be a cytokine induced killer cell.

The cell may be derived from a patient's own peripheral blood (1st party), or in the setting of a haematopoietic stem cell transplant from donor peripheral blood (2nd party), or peripheral blood from an unconnected donor (3rd party). T or NK cells, for example, may be activated and/or expanded prior to being transduced with nucleic acid molecule(s) encoding the polypeptides of the invention, for example by treatment with an anti-CD3 monoclonal antibody.

Alternatively, the cell may be derived from ex vivo differentiation of inducible progenitor cells or embryonic progenitor cells to T cells. Alternatively, an immortalized T-cell line which retains its lytic function may be used.

The cell may be a haematopoietic stem cell (HSC). HSCs can be obtained for transplant from the bone marrow of a suitably matched donor, by leukapheresis of peripheral blood after mobilization by administration of pharmacological doses of cytokines such as G-CSF [peripheral blood stem cells (PBSCs)], or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen.

Engineered Protein

As used herein, “engineered protein” refers to the protein which the immune cell has been engineered to express. The engineered protein may comprise at least two target-binding domains coupled to an intracellular retention signal.

The engineered protein may comprise one polypeptide chain or more than one polypeptide chain, for example at least two, or at least three, or at least four, or at least five or more polypeptide chains.

The engineered protein may comprise one, two, three, four, five or more polypeptide chains.

Suitably the at least two target-binding domains may be physically coupled to the intracellular retention signal.

The at least two target-binding domains may connected or interconnected to the intracellular retention signal. In one aspect, the at least two target-binding domains may be connected to or with the intracellular retention signal. In other words, the engineered protein may comprise one polypeptide chain which comprises at least two target-binding domains and an intracellular retention signal.

The at least two target-binding domains may be connected directly or indirectly to the intracellular retention signal. A first target-binding domain may be connected directly to the intracellular retention signal and a second target-binding domain may be indirectly connected to the intracellular retention signal, for example the second target-binding domain may be connected to the intracellular retention signal via the first target-binding domain or via a linker. In one aspect, at least one of the at least two target-binding domains is directly connected to the intracellular retention signal. Suitably, the at least two target-binding domains may be linked in series to the intracellular retention signal wherein one of the target-binding domains is directly connected to the intracellular retention signal.

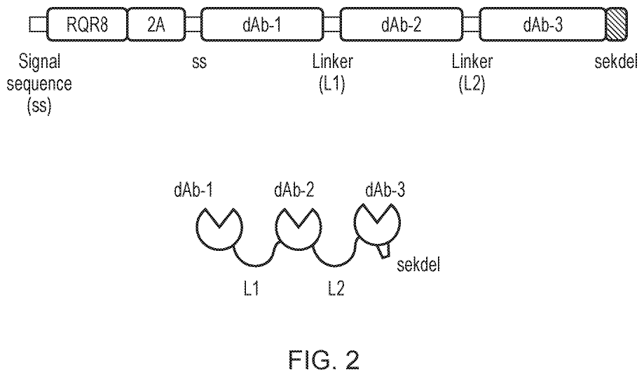

See for example FIG. 2 in which at least two target-binding domains (dAb-1, dAb-2 and dAb-3) are connected to each other by linkers and one target-binding domain (dAb-3) is directly connected to the intracellular retention signal. In FIG. 2, three target-binding domains are coupled to an intracellular retention signal, wherein one target binding domain (dAb-3) is directly connected to the intracellular retention signal, and two target-binding domains (dAb-1 and Ab-2) are indirectly connected to said intracellular retention signal via linkers and a target-binding domain.

In another aspect, at least two of the target-binding domains may be directly connected to the intracellular retention signal.

A target-binding domain may be directly linked to the intracellular retention signal. For example, the engineered polypeptide may be encoded by a nucleic acid sequence which encodes at least two target-binding domains wherein at least one target-binding domain is directly in frame (e.g. without a linker) adjacent to a nucleic acid sequence encoding an intracellular retention signal.

A target-binding domain may be indirectly linked to the intracellular retention signal. For example, the nucleic acid sequence encoding at least two target-binding domains may be connected to a nucleic acid sequence encoding an intracellular retention signal through a linker, such as a peptide linker as described herein.

In one aspect, the at least two target binding domains may be connected to one another by a linker, such as a peptide linker. Suitably, the at least two target-binding domains may be coupled to the intracellular retention signal via linkers, preferably peptide linkers.

Numerous suitable linkers are known in the art which are suitable for connecting target-binding domains to the intracellular retention signal and/or for connecting target-binding domains to one another.

For example, non-naturally occurring peptides, such as a polypeptides comprising (or consisting) of hydrophilic residues of varying length, or a (GGGGS)n (SEQ ID NO: 9) or a polypeptide or a variant thereof, in which n is an integer of, e.g., about 3—about 12, inclusive, can be used according to the present invention. In some embodiments the linker comprises, GGGGSGGGGS (SEQ ID NO: 10) or a variant thereof. In particular embodiments, the linker comprises SGGGSGGGSGGGS (SEQ ID NO: 11) or a variant thereof.

Suitably, peptide linkers having lengths of about 5 to about 100 amino acids, inclusive, may be used in the present invention. Peptide linkers having lengths of about 20 to about 40 amino acids, inclusive, may be used in the present invention. Peptide linkers having lengths of at least 5 amino acids, at least 10 amino acids, at least 15 amino acids, at least 20 amino acids, at least 25 amino acids, at least 30 amino acids, at least 35 amino acids, or at least 40 amino acids may also be used in the present invention.

As would be appreciated by those of skill in the art, such linker sequences as well as variants of such linker sequences are known in the art. Methods of designing constructs that incorporate linker sequences as well as methods of assessing functionality are readily available to those of skill in the art.

In one aspect, at least two target-binding domains are coupled to the same intracellular retention signal in the same polypeptide chain of the engineered protein. Suitably, at least two may be at least three, or at least four or at least five target-binding domains.

In one aspect, the engineered protein consists of one polypeptide chain which comprises at least two target-binding domains which are coupled to the same intracellular retention signal. For example, FIG. 2 shows an engineered protein consisting of one polypeptide chain which comprises three target-binding domains (e.g. dAB-1, dAb-2 and dAb-3) which are coupled to the same intracellular retention signal (e.g. SEKDEL).

Suitably, the engineered protein may comprise a first peptide subunit comprising a first target-binding domain and an intracellular retention signal and a second peptide subunit comprising at least a second target-binding domain; wherein the first and second peptide subunits are coupled, preferably by a peptide linker or one or more disulphide bonds.

In one aspect, said at least two target-binding domains are coupled to the intracellular retention signal by at least one heteromultimeric protein. The heteromultimeric protein may comprise at least two, at least three, at least four heteromultimeric components. Suitably, the heteromultimeric protein may be heterodimer. Suitably, the heteromultimeric protein may be stabilised by disulphide bonds between the heteromltimeric components.

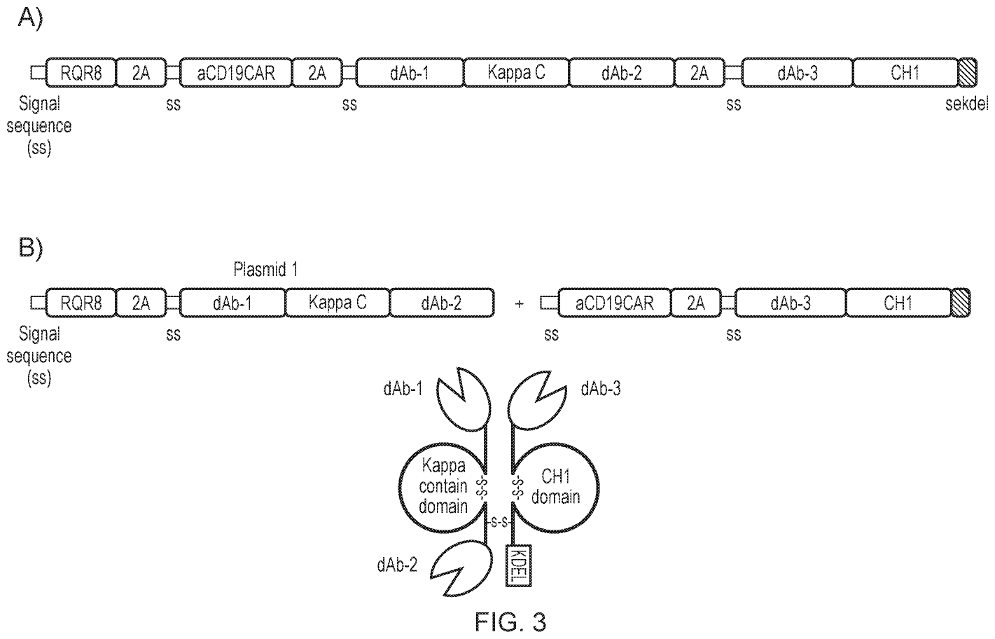

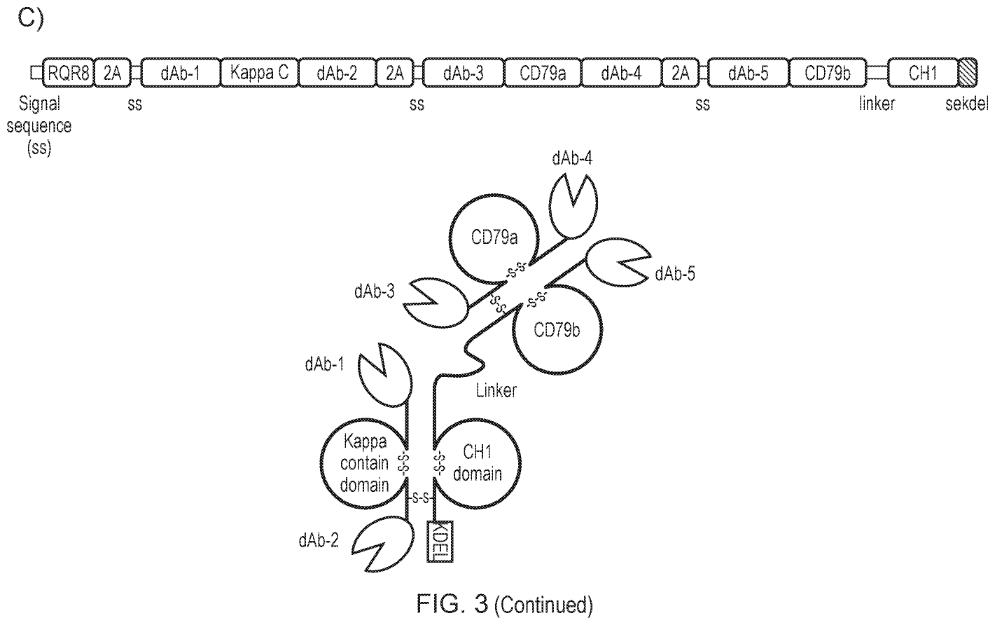

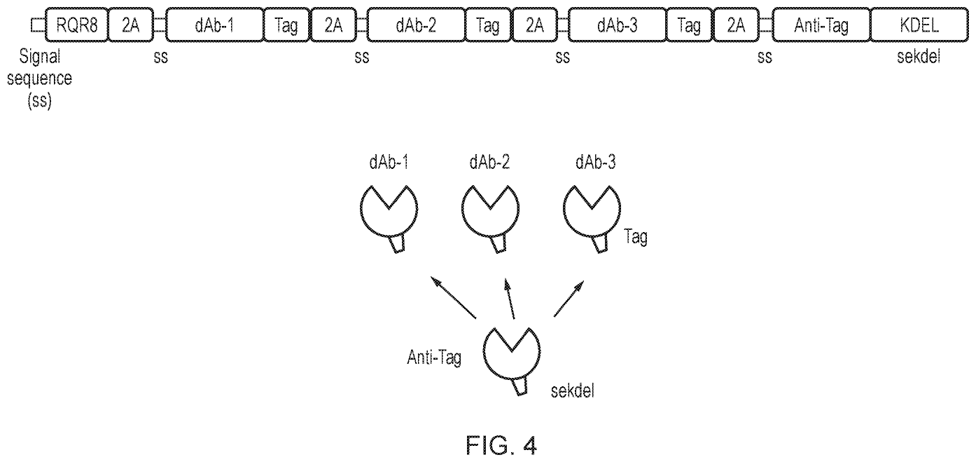

For example, FIG. 3c shows an engineered protein comprising two target-binding domains (dAb-3 and dAb-4) which are coupled to the intracellular retention signal by at least one heteromultimeric protein ((e.g. CD79a and CD79b). FIG. 3c also shows an engineered protein comprising two target-binding domains (dAb-1 and dAb-2) which are coupled to the intracellular retention signal (e.g. KDEL) by disulphide bond.

The heteromultimeric protein may be a stable heteromultimeric complex comprising at least a first and a second heteromultimer component. Suitably, the heteromultimeric protein may be a heterodimer pair.

The heteromultimeric protein may comprise a protein-protein interaction pair e.g. a first protein-interaction domain and a second protein-interaction domain. The first and second protein-interaction pairs are capable of associating to form a multimeric (e.g. dimer) complex. Suitably, the first and second-protein interaction pairs may be based on an epitope-tag system. For example, a heterodimeric pair may comprise a first protein interaction domain such as an epitope and a second protein interaction domain such as an epitope tag.

Below are examples of first and second heteromultimer components that associate to form a stable hetero-multimeric complex.

Suitably, the at least first and second-protein interaction pairs may be based on a naturally occurring multimeric protein or protein complex.

CD79a/CD79b

CD79 (cluster of differentiation 79) is protein that forms a complex with a B cell receptor and generates a signal following recognition of an antigen.

CD79 is composed of two distinct chains called CD79a and CD79b (formerly known as Ig-alpha and Ig-beta); these typically form a heterodimer on the surface of a B-cell stabilized by disulphide bonds. CD79a (UniProt: P11912) and CD79b (UniProt: P40259) are both members of the immunoglobulin superfamily.

Both CD79 chains contain an immunoreceptor tyrosine-based activation motif (ITAM) in their intracellular tails that they use to propagate a signal in a B cell, in a similar manner to CD3-generated signal transduction observed during T cell receptor activation on T cells.

A heteromultimeric protein may comprise the ectodomain from CD79a or CD79b. Exemplary sequences for these domains are given below, a heteromultimeric protein may comprise the flowing sequence or a variant thereof:

CD79a:
(SEQ ID No. 12)
LWMHKVPASLMVSLGEDAHFQCPHNSSNNANVTWWRVLH
GNYTWPPEFLGPGEDPNGTLIIQNVNKSHGGIYVCRVQE
GNESYQQSCGTYLRVRQPPPRPFLDMGEGTKNR
CD79b:
(SEQ ID NO. 13)
ARSEDRYRNPKGSACSRIWQSPRFIARKRGFTVKMHCYM
NSASGNVSWLWKQEMDENPQQLKLEKGRMEESQNESLAT
LTIQGIRFEDNGIYFCQQKCNNTSEVYQGCGTELRVMGF
STLAQLKQRNTLKD

An illustrative heteromultimeric arrangement is shown in FIG. 3c, where the target-binding domains dAb-3 and dAb-4 are coupled to the intracellular retention sequence (e.g. KDEL) via a heteromultimer comprising a CD79a ectodomain and a CD79b ectodomain

CH1 from IgG1/Kappa constant domain IgG1

IgG antibodies are multi-domain proteins with complex inter-domain interactions. Human IgG heavy chains associate with light chains to form mature antibodies capable of binding antigen. Light chains may be of the Kappa or gamma isotype.

The association of heavy and light constant domains forms a stable heterodimer. A heteromultimeric protein may comprise a heavy chain or a light chain constant region. The amino acid sequences for a Kappa chain constant region and a CH1 region from IgG1 are given below, but one skilled in the art will appreciate that many other suitable sequences from other antibodies are known.

Kappa chain:
(SEQ ID No. 14)
RTVAAPSVFIFPPSDEQLKSGTASVVCLLNNFYPREAKV
QWKVDNALQSGNSQESVTEQDSKDSTYSLSSTLTLSKAD
YEKHKVYACEVTHQGLSSPVTKSFNRGEC
CH1:
(SEQ ID NO. 15)
STKGPSVFPLAPSSKSTSGGTAALGCLVKDYFPEPVTVS
WNSGALTSGVHTFPAVLQSSGLYSLSSVVTVPSSSLGTQ
TYICNVNHKPSNTKVDKRV

An illustrative heteromultimeric protein may comprise a sequence as shown in SEQ ID NO: 12-15 or a variant thereof with at least 80% (such as at least 85%, at least 90%, at least 95%, at least 97%, at least 99%) identity to any of SEQ ID NO: 12-15 provided that the variant protein is capable of forming a heteromultimeric complex.

An illustrative heteromultimeric arrangement is shown in FIG. 3b, where the target-binding domains dAb-1 and dAb-2 are coupled to the intracellular retention sequence (e.g. KDEL) via a heteromultimer comprising a kappa containing domain and a CH1 domain.

The table below provides a non-limiting list of first and second heteromultimer components, including additional multimer pairs not described above. The first and second heteromultimer component pairs below may spontaneously associate to form a heteromultimer for use in the present invention:

| First multimer component | Second multimer component |
|---|---|
| CD79a (UniProt: P11912) | CD79b (UniProt: P40259) |
| Kappa Constant domain | CH1 from IgG1 |
| TRDC (UniProt: B7Z8K6) | TRGC (UniProt: P03986 or P03986) |
| CD1A (UniProt: P06126) | Beta-2-microglobulin (UniProt: P61760) |
| TRBC | TRAC |

A heteromultmeric protein may be formed from any two spontaneously associating pairs described in the above table. For example, the heteromultimeric protein may comprise both a CD79a/CD79b pair and a kappa containing domain/CH1 domain as shown in FIG. 3c.

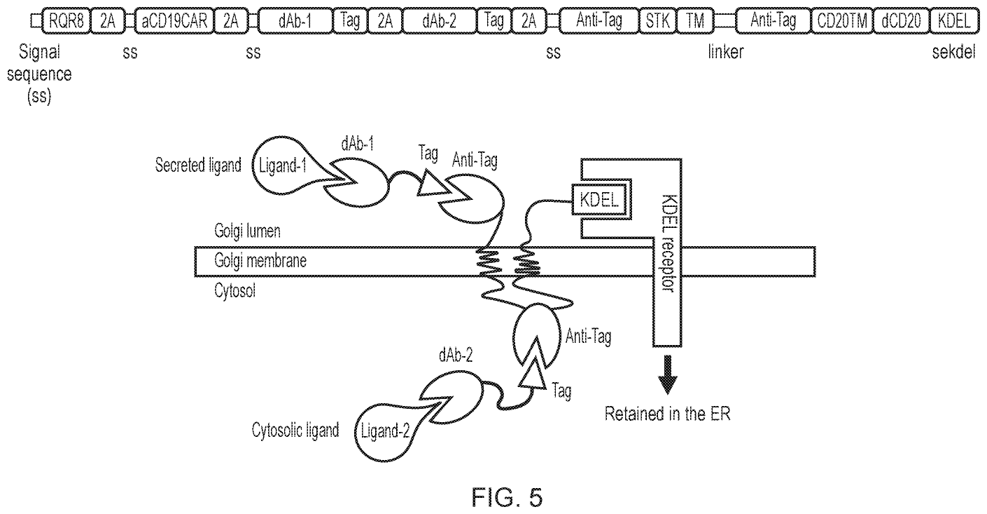

In one aspect, the at least one engineered protein comprises at least one transmembrane domain. Suitably, the engineered protein may comprise at least two transmembrane domains. The at least two target-binding domains may be located on different sides of the membrane which the transmembrane domain spans. Suitably, the engineered protein comprising a transmembrane domain may comprise target-binding domains which bind targets located in different cellular compartments. For example, FIG. 5 shows an engineered protein comprising a transmembrane domain, wherein the at least two target-binding domains are located in different cellular compartments. Suitably, at least one of the target-binding domains may bind a target which is a cytosolic protein. Suitably, at least one of the target-binding domains may bind a target which is an extracellular protein. Suitably, at least one of the target-binding domains may bind a target which is a transmembrane protein. Suitably, at least one of the target-binding domains may bind a target which is an intracellular protein.

A transmembrane domain may be derived from any transmembrane protein. For example, the transmembrane domain may be derived from human Tyrp-1 or human CD20. Exemplary transmembrane domains for use in the present invention include the following sequences and variants thereof having at least 80% (such as at least 85%, at least 90%, at least 95%, at least 96%, at least 99%) identity to SEQ ID NO: 16-17, provided that said variant functions as a transmembrane domain:

Tyrp-TM:
(SEQ ID NO: 16)
IIAIAVVGALLLVALIFGTASYLI
and
dCD20_TM:
(SEQ ID NO: 17)
IMNGLFHIALGGLLMIPAGIYA

In some aspects, an engineered protein additionally comprises a spacer domain. A spacer domain may be necessary to isolate the target-binding domain from the membrane and to allow it to assume a suitable orientation. A spacer domain may be necessary if the engineered protein comprises one or more transmembrane domains. FIG. 5 shows how spacer domains may be used to orientate target-binding domains.

A common spacer domain used is the Fc of IgG1. More compact spacers can suffice e.g. the stalk from CD8α and even just the IgG1 hinge alone, depending on the antigen.

Exemplary spacer domains include domains of STK and CD20. Sequences which may be used as spacer domains in the present invention include the following sequences and variants thereof having at least 80% (such as at least 85%, at least 90%, at least 95%, at least 96%, at least 99%) identity thereto:

CD8STK:
(SEQ ID NO: 18)
PTTTPAPRPPTPAPTIASQPLSLR
PEACRPAAGGAVHTRGLDFACDI
dCD20_N-terminal:
(SEQ ID NO: 19)
TTPRNSVNGTFPAEPMKGPIAMQSGPKP
LFRRMSSLVGPTQSFFMRESKTLGAVQ
dCD20_Short_Loop:
(SEQ ID NO: 20)
PICVTV

In one aspect, at least one target is an extracellular protein and at least one target is an intracellular protein; or
at least one target is a cytosolic protein and at least one target is an endoplasmic reticulum lumen protein.

Suitably, the at least first and second-protein interaction pairs may be based on a protein-protein interaction domains, such as epitope-tag systems.

In one aspect, each of the at least two target-binding domains and the intracellular retention signal are encoded as separate polypeptides; each of the polypeptides comprising the target-binding domains further comprises a first protein interaction domain and the polypeptide comprising the intracellular retention signal further comprises a second protein interaction domain wherein the first and second protein interaction domains are capable of binding to each other.

Suitably, at least one target-binding domain is connected to a first protein-interaction domain and the intracellular retention signal is connected to a second protein-interaction domain such that, when co-expressed in the cell, said first and second protein-interaction domains bind one another and the intracellular retention signal controls the cellular localisation of the target-binding domain and its target.

For example, FIG. 4 shows an illustrative embodiments in which target binding domains (e.g. dAb-1) connected to a first protein-interaction domain (e.g. tag) and the intracellular retention signal (e.g. KDEL) is connected to a second protein-interaction domain (anti-tag).

When co-expressed in the cell, the first and second protein interaction domains bind one another (tag-anti-tag interaction) and control the cellular localisation of the target domain (e.g. dAb-1) and its target (Ab-1).

In particular, FIG. 4 shows an illustrative embodiment in which at least two target-binding domains (e.g. dAb-1, dAB-2, dAb-3) and an intracellular retention signal (e.g. SEKDEL) encoded as separate polypeptides. Each of the polypeptides comprising a target-binding domain (e.g. dAb-1, dAB-2, dAb-3) further comprises a first protein interaction domain (tag) and the polypeptide comprising the intracellular retention signal (e.g. SEKDEL) further comprises a second protein interaction domain (e.g. anti-tag) wherein the first and second protein interaction domains are capable of binding to each other.

An exemplary heterodimeric pair (also referred to as a first and second protein interaction domain) is the ALFA peptide and the nanobody NbALFA.

Exemplary sequences which may be used in the present invention include the AFLA tag and anti-ALFA tag below, or variants thereof having at least 80% sequence identity thereto:

ALFA_tag:
(SEQ ID NO: 21)
PSRLEEELRRRLTEP
Anti-ALFA_Tag (NbALFA):
(SEQ ID NO: 22)
EVQLQESGGGLVQPGGSLRLSCTASGVTISALNAMAMGWYRQAPGE
RRVMVAAVSERGNAMYRESVQGRFTVTRDFTNKMVSLQMDNLKPED
TAVYYCHVLEDRVDSFHDYWGQGTQVTVSS

In one embodiment, the first and second protein-interaction domains may be an epitope tag system. As will be appreciated, specialised epitope tags are widely used for detecting, manipulating and purifying proteins.

Exemplary epitope tag systems which may be used in the present invention include the ALFA-tag, HA-tag (YPYDVPDYA—SEQ ID NO: 23), poly His-tag (His₃₋₁₀), FLAG-tag (DYKDDDDK—SEQ ID NO: 24), SPOT-tag (PDRVRAVSHWSS—SEQ ID NO: 25), EPEA/C-tag (EPEA—SEQ ID NO: 26) and myc-tag (EQKLISEEDL—SEQ ID NO: 27) systems.

The ALFA-tag forms a small and stable α-helix that is functional irrespective of its position on the target protein. The nanobody NbALFA binds ALFA-tagged proteins with low picomolar affinity.

In one embodiment the first and second protein-interaction domains may be an ALFA-tag system. In one embodiment a first-protein interaction domain may be the ALFA peptide and a second-protein interaction domain may bean anti-ALFA Dab.

Exemplary sequences which may be used in the present invention include the AFLA tag and anti-ALFA tag below, or variants thereof having at least 80% sequence identity thereto:

ALFA_tag:
(SEQ ID NO: 21)
PSRLEEELRRRLTEP
Anti-ALFA_Tag (NbALFA):
(SEQ ID NO: 22)
EVQLQESGGGLVQPGGSLRLSCTASGVTISALNAMAMGWYRQAPGE
RRVMVAAVSERGNAMYRESVQGRFTVTRDFTNKMVSLQMDNLKPED
TAVYYCHVLEDRVDSFHDYWGQGTQVTVSS

Intracellular Retention Signal

Protein targeting or protein sorting is the biological mechanism by which proteins are transported to the appropriate destinations in the cell or outside of it. Proteins can be targeted to the inner space of an organelle, different intracellular membranes, plasma membrane, or to exterior of the cell via secretion. This delivery process is carried out based on sequence information contain in the protein itself.

Proteins synthesised in the rough endoplasmic reticulum (ER) of eukaryotic cells use the exocytic pathway for transport to their final destinations. Proteins lacking special sorting signals are vectorially transported from the ER via the Golgi and the trans-Golgi network (TGN) to the plasma membrane. Other proteins have targeting signals for incorporation into specific organelles of the exocytic pathway, such as endosomes and lysosomes.

Lysosomes are acidic organelles in which endogenous and internalised macromolecules are degraded by luminal hydrolases. Endogenous macromolecules reach the lysosome by being sorted in the TGN from which they are transported to endosomes and then lysosomes.

The targeting signals used by a cell to sort proteins to the correct intracellular location may be exploited by the present invention. The signals may be broadly classed into the following types:
i) endocytosis signals
ii) Golgi retention signals
iii) TGN recycling signals
iv) ER retention signals
v) lysosomal sorting signals

The intracellular retention signal may direct the transmembrane protein away from the secretory pathway during translocation from the ER.

The intracellular retention signal may direct the transmembrane protein to an intracellular compartment or complex. The intracellular retention signal may direct the transmembrane protein to a membrane-bound intracellular compartment.

For example, the intracellular retention signal may direct the protein to a lysosomal, endosomal or Golgi compartment (trans-Golgi Network, ‘TGN’).

Within a normal cell, proteins arising from biogenesis or the endocytic pathway are sorted into the appropriate intracellular compartment following a sequential set of sorting decisions. At the plasma membrane, proteins can either remain at the cell surface or be internalised into endosomes. At the TGN, the choice is between going to the plasma membrane or being diverted to endosomes. In endosomes, proteins can either recycle to the plasma membrane or go to lysosomes. These decisions are governed by sorting signals on the proteins themselves.

Lysosomes are cellular organelles that contain acid hydrolase enzymes that break down waste materials and cellular debris. The membrane around a lysosome allows the digestive enzymes to work at the pH they require. Lysosomes fuse with autophagic vacuoles (phagosomes) and dispense their enzymes into the autophagic vacuoles, digesting their contents.

An endosome is a membrane-bounded compartment inside eukaryotic cells. It is a compartment of the endocytic membrane transport pathway from the plasma membrane to the lysosome and provides an environment for material to be sorted before it reaches the degradative lysosome. Endosomes may be classified as early endosomes, late endosomes, or recycling endosomes depending on the time it takes for endocytosed material to reach them. The intracellular retention signal used in the present invention may direct the protein to a late endosomal compartment.

The Golgi apparatus is part of the cellular endomembrane system, the Golgi apparatus packages proteins inside the cell before they are sent to their destination; it is particularly important in the processing of proteins for secretion.

There is a considerable body of knowledge which has arisen from studies investigating the sorting signals present in known proteins, and the effect of altering their sequence and/or position within the molecule (Bonifacino and Traub (2003) Ann. Rev. Biochem. 72:395-447; Braulke and Bonifacino (2009) Biochimica and Biophysica Acta 1793:605-614; Griffith (2001) Current Biology 11:R226-R228; Mellman and Nelson (2008) Nat Rev Mol Cell Biol. 9:833-845; Dell'Angelica and Payne (2001) Cell 106:395-398; Schafer et al (1995) EMBO J. 14:2424-2435; Trejo (2005) Mol. Pharmacol. 67:1388-1390). Numerous studies have shown that it is possible to insert one or more sorting signals into a protein of interest in order to alter the intracellular location of a protein of interest (Pelham (2000) Meth. Enzymol. 327:279-283).

Examples of endocytosis signals include those from the transferrin receptor and the asialoglycoprotein receptor.

Examples of signals which cause TGN-endosome recycling include those form proteins such as the Cl- and CD-MPRs, sortilin, the LDL-receptor related proteins LRP3 and LRP10 and β-secretase, GGA1-3, LIMP-II, NCP1, mucolipn-1, sialin, GLUT8 and invariant chain.

Examples of TGN retention signals include those from the following proteins which are localized to the TGN: the prohormone processing enzymes furin, PC7, CPD and PAM; the glycoprotein E of herpes virus 3 and TGN38.

Examples of ER retention signals include C-terminal signals such as KDEL, KKXX or KXKXX and the RXR(R) motif of potassium channels. Known ER proteins include the adenovirus E19 protein and ERGIC53.

Examples of lysosomal sorting signals include those found in lysosomal membrane proteins, such as LAMP-1 and LAMP-2, CD63, CD68, endolyn, DC-LAMP, cystinosin, sugar phosphate exchanger 2 and acid phosphatase.

The engineered immune cell of the present invention comprises at least one engineered protein which comprises at least two target-binding domains coupled to an intracellular retention signal.

Intracellular retention signals are well known in the art (see, for example, Bonifacino & Traub; Annu. Rev. Biochem.; 2003; 72; 395-447).

The present invention also provides a nucleic acid construct which comprises the following structure:

A-X-B-C
in which:
A and B are nucleic acid sequences encoding target-binding domain as defined herein; and X is a linker as defined herein; and C is an intracellular retention signal as defined herein.

Suitably, “intracellular retention signal” refers to an amino acid sequence which directs or maintains the protein in which it is encompassed to a cellular compartment other than that to which it would be directed in the absence of the intracellular retention signal. Suitably, the intracellular retention signal directs or maintains the protein in which it is encompassed to a cellular compartment other than the cell surface membrane or to the exterior of the cell.

The intracellular retention signal may be any protein or protein domain which is a resident of a given intracellular compartment. This means that said protein or domain is in majority, located in a given compartment. At least 80%, 85%, 90%, 95%, 96%, 97%, 98% or 99% of said protein or domain is located in said compartment in a cell. The intracellular retention signal prevents an engineered protein according to the present invention from being secreted from the cell or from being translocated to the plasma membrane.

As used herein “compartment” or “subcellular compartment” refers to a given subdomain of cell. A compartment may be an organelle (such as endoplasmic reticulum, Golgi apparatus, endosome, lysosome) or an element of an organelle (such as multi-vesicular bodies of endosomes, cis-medial- or trans-cisternae of the Golgi apparatus etc.) or the plasma membrane or sub-domains of the plasma membrane (such as apical, basolateral, axonal domains) or micro domains such as focal adhesions or tight junctions.

An “intracellular compartment” refers to a compartment within a cell.

According to the present invention, at least two target proteins may be retained within the cell or within a specific intracellular compartment by an interaction with a cognate target binding domain which is itself coupled to an intracellular retention signal. The at least two target proteins may be retained within different intracellular compartments.

In one aspect, the intracellular retention signal directs the protein to a Golgi (trans-Golgi Network, “TGN”), endosomal or lysosomal compartment.

In one aspect, the intracellular retention signal is selected from the following group: a Golgi retention sequence; a trans-Golgi network (TGN) recycling signal; an endoplasmic reticulum (ER) retention sequence; a proteasome localization sequence or a lysosomal sorting signal. The intracellular retention signal may be a protein or domain which is resident in the Golgi. Suitably, the Golgi retention domain may be selected from the group comprising: Giantin (GolgB1, GenBank Accession number NM+004487.3), TGN38/46, Menkes receptor and Golgi enzymes such as ManII (α-1,3-1,6 mannosidase, Genbank accession number NM_008549), Sialyl Transferase (β-galactosamide α2,6-sialytransferae 1, NM_003032), GaIT (β-1,4-galactosyltransferase 1, NM_001497) adenoviral E19, HLA invariant chain or fragments thereof comprising the localisation domains.

In one aspect the Golgi retention sequence comprises an amino acid sequence selected from: SEKDEL(SEQ ID NO: 1), KDEL (SEQ ID NO: 2), KKXX (SEQ ID NO: 3), KXKXX (SEQ ID NO: 4), a tail of adenoviral E19 protein comprising the sequence KYKSRRSFIDEKKMP (SEQ ID NO: 5), a fragment of HLA invariant chain comprising the sequence MHRRRSRSCR (SEQ ID NO: 6), KXD/E (SEQ ID NO: 7) or a YQRL (SEQ ID NO: 8) or variants thereof having at least 80% sequence identity thereto which retain the ability to function as Golgi retention sequences, wherein X is any amino acid.

Suitably, the retention signal may be a SEKEDL (SEQ ID NO: 1) or KDEL (SEQ ID NO: 2) sequence. The KDEL receptor binds protein in the ER-Golgi intermediate compartment, or in the early Golgi and returns them to the ER. Proteins only leave the ER after the KDEL sequence has been cleaved off. Thus the protein resident in the ER will remain in the ER as long as it contains a KDEL sequence. Although the common mammalian signal is KDEL, it has been shown that the KDEL receptor binds the sequence HDEL more tightly (Scheel et al; J. Biol. Chem. 268; 7465 (1993)). The intracellular retention signal may be HDEL.

Suitably, the retention domain—in particular a Golgi retention sequence such as SEKDEL(SEQ ID NO: 1) or KDEL (SEQ ID NO: 2)—is located at the C-terminus of the engineered protein to be targeted to a particular intracellular compartment, in particular the Golgi.

Suitably, the retention domain—in particular a Golgi retention sequence such as SEKDEL (SEQ ID NO: 1) or KDEL (SEQ ID NO: 2)—is not located immediately upstream/5′ of a self-cleaving peptide (such as a 2A or 2A-like peptide) in a nucleic acid construct of the invention.

KKX∝X∝ and KX′KX′X′ signals are retrieval signals which can be placed on the cytoplasmic side of a type I membrane protein. Sequence requirements of these signals are provided in detail by Teasdale & Jackson (Annu. Rev. Cell Dev. Biol.; 12; 27 (1996)).

Suitably, the retention signal may be a KKXX (SEQ ID NO: 3) motif. Suitably the KKXX domain may be located that the C terminus of the protein. KKXX is responsible for retrieval of ER membrane proteins from the cis end of the Golgi apparatus by retrograde transport, via interaction with the coat protein (COPI) complex.

Suitably, the retention signal may be a KXKXX (SEQ ID NO: 4) motif.

The intracellular retention signal may be from the adenovirus E19 protein. The intracellular retention signal may be from the protein E3/19K, which is also known as E3gp 19 kDa; E19 or GP19K. The intracellular retention signal may comprise the full cytosolic tail of E3/19K, which is shown as SEQ ID No. 5; or the last 6 amino acids of this tail, which is shown as SEQ ID No. 28. Suitably, the retention signal may be a tail of adenoviral E19 protein comprising the sequence KYKSRRSFIDEKKMP (SEQ ID NO: 5). Suitably, the retention signal may be a tail of adenoviral E19 protein comprising the sequence SEQ ID No. 28: DEKKMP.

Suitably, the retention domain may be an N-terminal fragment of the invariant chain of HLA comprising the sequence MHRRRSRSCR (SEQ ID NO: 6) or a variant thereof having at least 80% identity thereto and which retains the ability to function as a retention signal. The retention signal may be a protein or domain which is resident in the ER.

The ER retention signal may selected from the group comprising: an isoform of the invariant chain which resides in the ER (li33), Ribophorin I, Ribophorin II, SEC61 or cytochrome b5 or fragments thereof comprising the localisation domains. An example of an ER localisation domain is the ER localisation of Ribophorin II, Genbank accession BC060556.1.

In one aspect, the endoplasmic reticulum retention signal is selected from: Ribophorin I, Ribophorin II, SEC61 or cytochrome b5.

The intracellular retention signal may be a tyrosine-based sorting signal, a dileucine-based sorting signal, an acidic cluster signal, a lysosomal avoidance signal, an NPFX′(1,2)D-Type signal, a KDEL, a KKX′X′ or a KX′KX′X′ signal (wherein X′ is any amino acid).

Tyrosine-based sorting signals mediate rapid internalization of transmembrane proteins from the plasma membrane and the targeting of proteins to lysosomes (Bonifacino & Traub; supra). Two types of tyrosine-based sorting signals are represented by the NPX′Y and YX′X′Z′ consensus motifs (wherein Z′ is an amino acid with a bulky hydrophobic side chain).

NPX′Y signals have been shown to mediate rapid internalization of type I transmembrane proteins, they occur in families such as members of the LDL receptor, integrin β, and β-amyloid precursor protein families.

Examples of NPX′Y signals are provided in Table 1.

TABLE 1
NPX′Y signals

| Protein | Species | Sequence |
|---|---|---|
| LDL receptor | Human | Tm-10-INFDNPVYQKTT-29 |
| LRP1 (1) | Human | Tm-21-VEIGNPTYKMYE-64 |
| LRP1 (2) | Human | Tm-55-TNFTNPVYATLY-33 |
| LRP1 | Drosophila | Tm-43-GNFANPVYESMY-38 |
| LRP1 (1) | C. elegans | Tm-54-TTFTNPVYELED-91 |
| LRP1 (2) | C. elegans | Tm-140-LRVDNPLYDPDS-4 |
| Megalin (1) | Human | Tm-70-IIFENPMYSARD-125 |
| Megalin (2) | Human | Tm-144-TNFENPIYAQME-53 |
| Integrin 13-1 (1) | Human | Tm-18-DTGENPIYKSAV-11 |
| Integrin 13-1 (2) | Human | Tm-30-TTVVNPKYEGK |
| Integrin 13 (1) | Drosophila | Tm-26-WDTENPIYKQAT-11 |
| Integrin 13 (2) | Drosophila | Tm-35-STFKNPMYAGK |
| APLP1 | Human | Tm-33-HGYENPTYRFLE-3 |
| APP | Human | Tm-32-NGYENPTYKFFE-4 |
| APP-like | Drosophila | Tm-38-NGYENPTYKYFE-3 |
| Insulin receptor | Human | Tm-36-YASSNPEYLSAS-379 |
| EGR receptor (1) | Human | Tm-434-GSVQNPVYHNQP-96 |
| EGR receptor (2) | Human | Tm-462-TAVGNPEYLNTV-68 |
| EGR receptor (3) | Human | Tm-496-ISLDNPDYQQDF-34 |

Numbers in parentheses indicate motifs that are present in more than one copy within the same protein.
The signals in this and other tables should be considered examples.
Key residues are indicated in bold type.
Numbers of amino acids before (i.e., amino-terminal) and after (i.e., carboxy-terminal) the signals are indicated.
Abbreviations: Tm, transmembrane; LDL, low density lipoprotein; LRP1, LDL receptor related protein 1; APP, 13-amyloid precursor protein; APLP1, APP-like protein 1.

YX′X′Z′-type signals are found in endocytic receptors such as the transferrin receptor and the asialoglycoprotein receptor, intracellular sorting receptors such as the Cl- and CD-MPRs, lysosomal membrane proteins such as LAMP-1 and LAMP-2, and TGN proteins such as TGN38 and furin, as well as in proteins localized to specialized endosomal-lysosomal organelles such as antigen-processing compartments (e.g., HLA-DM) and cytotoxic granules (e.g., GMP-17). The YX′X′Z′-type signals are involved in the rapid internalization of proteins from the plasma membrane. However, their function is not limited to endocytosis, since the same motifs have been implicated in the targeting of transmembrane proteins to lysosomes and lysosome-related organelles.

Examples of YX′X′Z′-type signals are provided in Table 2.

TABLE 2
YX′X′Z′-type signals

| Protein | Species | Sequence |
|---|---|---|
| LAMP-1 | Human | Tm-RKRSHAGYQTI |
| LAMP-2a | Human | Tm-KHHHAGYEQF |
| LAMP-2a | Chicken | Tm-KKHHNTGYEQF |
| LAMP-2b | Chicken | Tm-RRKSRTGYQSV |
| LAMP-2c | Chicken | Tm-RRKSYAGYQTL |
| LAMP | Drosophila | Tm-RRRSTSRGYMSF |
| LAMP | Earthworm | Tm-RKRSRRGYESV |
| CD63 | Human | Tm-KSIRSGYEVM |
| GMP-17 | Human | Tm-HCGGPRPGYETL |
| GMP-17 | Mouse | Tm-HCRTRRAEYETL |
| CD68 | Human | Tm-RRRPSAYQAL |
| CD1b | Human | Tm-RRRSYQNIP |
| CD1c | Human | Tm-KKHCSYQDIL |
| CD1d | Mouse | Tm-RRRSAYQDIR |
| CD1 | Rat | Tm-RKRRRSYQDIM |
| Endolyn | Rat | Tm-KFCKSKERNYHTL |
| Endolyn | Drosophila | Tm-KFYKARNERNYHTL |
| TSC403 | Human | Tm-KIRLRCQSSGYQRI |
| TSC403 | Mouse | Tm-KIRQRHQSSAYQRI |
| Cystinosin | Human | Tm-HFCLYRKRPGYDQLN |
| Putative solute carrier | Human | Tm-12-SLSRGSGYKEI |
| TRP-2 | Human | Tm-RRLRKGYTPLMET-11 |
| HLA-DM [symbol] | Human | Tm-RRAGHSSYTPLPGS-9 |
| LmpA | Dictyosteliu | Tm-KKLRQQKQQGYQAIINNE |
| Putative lysosomal | Dictyosteliu | Tm-RSKSNQNQSYNLIQL |
| LIMP-II | Dictyosteliu | Tm-RKTFYNNNQYNGYNIIN |
| Transferrin receptor | Human | 16-PLSYTRFSLA-35-Tm |
| Asialoglycoprotein | Human | MTKEYQDLQHL-29-Tm |
| CI-MPR | Human | Tm-22-SYKYSKVNKE-132 |
| CD-MPR | Human | Tm-40-PAAYRGVGDD-16 |
| CTLA-4 | Human | Tm-10-TGVYVKMPPT-16 |
| Furin | Human | Tm-17-LISYKGLPPE-29 |
| TGN38 | Rat | Tm-23-ASDYQRLNLKL |
| gp41 | HIV-1 | Tm-13-RQGYSPLSFQT-144 |
| Acid phosphatase | Human | Tm- |
| | | RMQAQPPGYRHVADGEDHA |

Dileucine-based sorting signals ([DE]X′X′X′LL[LI]) play critical roles in the sorting of many type I, type II, and multispanning transmembrane proteins. Dileucine-based sorting signals are involved in rapid internalization and lysosomal degradation of transmembrane proteins and the targeting of proteins to the late endosomal-lysosomal compartments. Transmembrane proteins that contain constitutively active forms of this signal are mainly localised to the late endosomes and lysosomes.

Examples of [DE]X′X′X′LL[LI] sorting signals are provided in Table 3.

TABLE 3
[DE]X′X′X′LL[LI] sorting signals

| Protein | Species | Signal |
|---|---|---|
| CD3-γ | Human | Tm-8-SDKQTLLPN-26 |
| LIMP-II | Rat | Tm-11-DERAPLIRT |
| Nmb | Human | Tm-37-QEKDPLLKN-7 |
| QNR-71 | Quail | Tm-37-TERNPLLKS-5 |
| Pmel17 | Human | Tm-33-GENSPLLSG-3 |
| Tyrosinase | Human | Tm-8-EEKQPLLME-12 |
| Tyrosinase | Medaka | Tm-16-GERQPLLQS-13 |
| | fish | |
| Tyrosinase | Chicken | Tm-8-PEIQPLLTE-13 |
| TRP-1 | Goldfish | Tm-7-EGRQPLLGD-15 |
| TRP-1 | Human | Tm-7-EANQPLLTD-20 |
| TRP-1 | Chicken | Tm-7-ELHQPLLTD-20 |
| TRP-2 | Zebrafish | Tm-5-REFEPLLNA-11 |
| VMAT2 | Human | Tm-6-EEKMAILMD-29 |
| VMATI | Human | Tm-6-EEKLAILSQ-32 |
| VAchT | Mouse | Tm-10-SERDVLLDE-42 |
| VAMP4 | Human | 19-SERRNLLED-88-Tm |
| Neonatal | Rat | Tm-16-DDSGDLLPG-19 |
| FcR | | |
| CD4 | Human | Tm-12-SQIKRLLSE-17 |
| CD4 | Cat | Tm-12-SHIKRLLSE-17 |
| GLUT4 | Mouse | Tm-17-RRTPSLLEQ-17 |
| GLUT4 | Human | Tm-17-HRTPSLLEQ-17 |
| IRAP | Rat | 46-EPRGSRLLVR-53-Tm |
| Ii | Human | MDDQRDLISNNEQLPMLGR-11-Tm |
| Ii | Mouse | MDDQRDLISNHEQLPILGN-10-Tm |
| Ii | Chicken | MAEEQRDLISSDGSSGVLPI-12-Tm |
| Ii-1 | Zebrafish | MEPDHQNESLIQRVPSAETILGR-12-Tm |
| Ii-2 | Zebrafish | MSSEGNETPLISDQSSVNMGPQP-8-Tm |
| Lamp | Trypanosome | Tm-RPRRRTEEDELLPEEAEGLIDPON |
| Menkes | Human | Tm-74-PDKHSLLVGDFREDDDTAL |
| protein | | |
| NPC1 | Human | Tm-13-TERERLLNF |
| AQP4 | Human | Tm-32-VETDDLIL-29 |
| RME-2 | C. elegans | Tm-104-FENDSLL |
| Vam3p | S. cerevisiae | 153-NEQSPLLHN-121-Tm |
| ALP | S. cerevisiae | 7-SEQTRLVP-18-Tm |
| Gaplp | S. cerevisiae | Tm-23-EVDLDLLK-24 |

DX′X′LL signals constitute a distinct type of dileucine-based sorting signals. These signals are present in several transmembrane receptors and other proteins that cycle between the TGN and endosomes, such as the Cl- and CD-MPRs, sortilin, the LDL-receptor-related proteins LRP3 and LRP10, and β-secretase.

Examples of DX′X′LL sorting signals are provided in Table 4.

TABLE 4
DX′X′LL sorting signals

| Protein | Species | Sequence |
|---|---|---|
| CI-MPR | Human | Tm-151-SFHDDSDEDLLHI |
| CI-MPR | Bovine | Tm-150-TFHDDSDEDLLHV |
| CI-MPR | Rabbit | Tm-151-SFHDDSDEDLLNI |
| CI-MPR | Chicken | Tm-148-SFHDDSDEDLLNV |
| CD-MPR | Human | Tm-54-EESEERDDHLLPM |
| CD-MPR | Chicken | Tm-54-DESEERDDHLLPM |
| Sortilin | Human | Tm-41-GYHDDSDEDLLE |
| SorLA | Human | Tm-41-ITGFSDDVPMVIA |
| Head- | Hydra | Tm-41-INRFSDDEPLVVA |
| activator | | |
| BP | | |
| LRP3 | Human | Tm-237-MLEASDDEALLVC |
| ST7 | Human | Tm-330-KNETSDDEALLLC |
| LRP10 | Mouse | Tm-235-WVVEAEDEPLLA |
| LRP10 | Human | Tm-237-WVAEAEDEPLLT |
| Beta- | Human | Tm-9-HDDFADDISLLK |
| secretase | | |
| Mucolipin-1 | Mouse | Tm-43-GRDSPEDHSLLVN |
| Nonclassical | Deer | Tm-6-VRCHPEDDRLLG |
| MHC-I | mouse | |
| FLJ30532 | Human | Tm-83-HRVSQDDLDLLTS |
| GGA1 | Human | 350-ASVSLLDDELMSL-275 |
| GGAI | Human | 415-ASSGLDDLDLLGK-211 |
| GGA2 | Human | 408-VQNPSADRNLLDL-192 |
| GGA3 | Human | 384-NALSWLDEELLCL-326 |
| GGA | Drosophila | 447-TVDSIDDVPLLSD-116 |

Another family of sorting motifs is provided by clusters of acidic residues containing sites for phosphorylation by CKII. This type of motif is often found in transmembrane proteins that are localized to the TGN at steady state, including the prohormone-processing enzymes furin, PC6B, PC7, CPD, and PAM, and the glycoprotein E of herpes virus 3.

Examples of acidic cluster signals are provided in Table 5.

TABLE 5
Acidic cluster sorting signals

| Protein | Species | Sequence |
|---|---|---|
| Furin | Mouse | Tm-31-QEECPSDSEEDEG-14 |
| PC6B (1)^{a} | Mouse | Tm-39-RDRDYDEDDEDDI-36 |
| PC6B (2) | Mouse | Tm-69-LDETEDDELEYDDES-4 |
| PC7 | Human | Tr-38-KDPDEVETES-47 |
| CPD | Human | Tm-36-HEFQDETDTERET-6 |
| PAM | Human | Tm-59-QEKEDDGSESCEEY-12 |
| VMAT2 | Human | Tm-35-GEDEESESD |
| VMAT1 | Human | Tm-35-GEDSDEEPDHEE |
| VAMP4 | Human | 25-LEDDSDEEEDF-81-Tm |
| Glycoprotein B | HCMV | Tm-125-KDSDEEENV |
| Glycoprotein | Herpes virus | Tr-28-FEDSESTDTECEF-21 |
| | 3 | |
| E Nef | HIV-1 (AAL65476) | 55-LEAQEEEEV-139 |
| Kexip (1) | S. | Tm-29-ADDLESGLGAEDDLEQDEQLEG-40 |
| | cerevisiae | |
| Kex1p (2) | S. | Tm-79-TEIDESFEMTDF |
| | cerevisise | |
| Kex2p | S. | Tm-36-TEPEEVEDFDFDLSDEDH-61 |
| | cerevisiae | |
| Vps10p | S. | Tm-112-FEIEEDDVPILEEEH-37 |
| | cerevisiae | |

The intracellular retention signal may be selected from the group of: NPX′Y, YX′X′Z, [DE]X′X′XL[LI], DX′X′LL, DP[FW], FX′DX′F, NPF, LZX′Z[DE], LLDLL, PWDLW, KDEL, HDEL, KKX′X′ or KX′KX′X′; wherein X′ is any amino acid and Z′ is an amino acid with a bulky hydrophobic side chain.

The intracellular retention signal may be any sequence shown in Tables 1 to 5.

The intracellular retention signal may comprise the Tyrosinase-related protein (TYRP)-1 intracellular retention signal. The intracellular retention signal may comprise the TYRP-1 intracellular domain. The intracellular retention signal may comprise the sequence NQPLLTD (SEQ ID No. 29) or a variant thereof.

TYRP1 is a well-characterized melansomal protein which is retained in the melanosome (a specialized lysosome) at >99% efficiency. TYRP1 is a 537 amino acid transmembrane protein with a lumenal domain (1-477aa), a transmembrane domain (478-501), and a cytoplasmic domain (502-537). A di-leucine signal residing on the cytoplasmic domain causes retention of the protein. This di-leucine signal has the sequence shown as SEQ ID No. 29 (NQPLLTD).

Target Binding Domain

A target binding domain may be a protein or polypeptide chain which is capable of binding to a specific target molecule (or target protein) whose cellular localisation is to be controlled.

In one aspect, at least one target is selected from: a cytosolic protein, an intracellular protein, an extracellular protein, and a transmembrane protein.

Suitably, the target may be an endogenous protein. For example, the target may be a protein which is naturally expressed by the cell. In other words the cell has not been engineered to express the target.

In one aspect, the target binding domain may be a protein-protein-interaction domain. Suitably, the target binding domain may comprise a protein interaction domain.

In one aspect, the target binding domain comprises an antibody, an antibody fragment or antigen binding fragment, a single-chain variable fragment (scFv), a domain antibody (dAb), a single domain antibody (sdAb), a VHH/nanobody, a nanobody, an affibody, a fibronectin artificial antibody scaffold, an anticalin, an affilin, a DARPin, a VNAR, an iBody, an affimer, a fynomer, an abdurin/nanoantibody, a centyrin, an alphabody or a nanofitin which binds to a target.

In one aspect, at least one target-binding domain is a domain antibody (dAb).

In one aspect at least one target-binding domain is a single-chain variable fragment (scFv).

In one aspect, the target-binding domain may be a receptor or a ligand that binds to a target molecule. For example, the target may be PD-1 and the target-binding molecule may be a ligand that binds PD-1 (e.g., PD-L1 or PD-L2).

The target may be any protein which it is desirable to control the localisation of, for example, for which it is desirable to control (e.g. reduce or inhibit) secretion of. It may be desirable to control (e.g. reduce or inhibit) the secretion of proteins which modulate the tumour environment e.g. immunomodulatory cytokines such as interleukin 12 (IL-12), or proteins which cause inflammation.

Alternatively, the target may be any protein which it is desirable to control the cell surface expression of. For example it may be desirable control (e.g. to reduce or inhibit) the expression of cell surface proteins to reduce fratricide when if a CAR T cell is targeting a group of ligands also expressed on the surface of said CAR T cell (e.g. CD2, CD5 or CD7).

It may also be desirable to control (e.g. reduce or inhibit) the expression of inhibitory proteins which are typically present at the surface of cells (such as PD1, TIGIT, BTLA, TIM3, Fas, CTLA, TBR2 or LAG3).

In some cases, the target may be a protein for which it is desirable to control the intracellular cellular localisation of. It may be desirable to control the localisation of proteins in specific cellular compartments to abrogate their function. For example, the cellular localisation of cytosolic proteins whose function is dependent on plasma membrane location (e.g. ZAP70, SLP76 or AKT).

In one aspect, the at least two target-binding domains may bind to different regions of the same target.

Alternatively, the at least two target-binding domains may bind to different targets. Suitably, the at least two targets may be localised in the same cellular compartment. Suitably, the at least two targets may be localised in different cellular compartments.

In one aspect, at least one target-binding domain binds to a component of a CD3/T-cell receptor (TCR) complex, a cytokine, a human leukocyte antigen (HLA) class I molecule, a receptor that downregulates immune response, a ligand expressed on T cells, or a cytosolic proteins that modulate the immune response.

Suitably, the component in a CD3/TCR complex may be CD3ε, TCRα, TCRαβ, TCRγ, TCRδ, CD3δ, CD3γ, or CD3ζ.

An exemplary target binding domain which may be used in the present invention is anti-CD3E UCHT (shown as Ab-1 in FIG. 2) or a variant thereof having at least 80% identity thereto:

Ab-1 (aCD3e_UCHT):
(SEQ ID NO: 30)
DIQMTQSPSSLSASVGNRVTITCRASQDIRNYLNWYQQKP
GKAPKLLIYYTSRLESGVPSRFSGSGSGTDYTLTISSLQP
EDFATYYCQQGNTLPWTFGQGTKVEIKSGGGGSGGGGSGG
GGSEVQLVESGGGLVQPGGSLRLSCAASGYSFTGYTMNWV
RQAPGKGLEWVALINPYKGVSTYNQKFKDRFTISVDKSKN
TAYLQMNSLRAEDTAVYYCARSGYYGDSDWYFDVWGQGTL
VTVSS

Suitably, the HLA Class I molecule may be B2-microglobulin, α1-microglobulin, α2-microglobulin, or α3-microglobulin.

An exemplary target binding domain which may be used in the present invention is anti-B2-microglobulin_dN6B2M (shown as Ab-2 in FIG. 2) or a variant thereof having at least 80% identity thereto:

Ab-2 (aB2M_dN6B2m):
(SEQ ID NO: 31)
QVQLQESGGGSVQAGGSLRLSCAASGYTDSRYCMAWFRQA
PGKEREWVARINSGRDITYYADSVKGRFTFSQDNAKNTVY
LQMDSLEPEDTATYYCATDIPLRCRDIVAKGGDGFRYWGQ
GTQVTVSS

Suitable, the target protein may be and MHC class II molecule. In humans the MHC class II protein complex is encoded by the human leukocyte antigen gene complex (HLA). HLAs corresponding to MHC class II are HLA-DP, HLA-DM, HLA-DOA, HLA-DOB, HLA-DQ and HLA-DR.

HLA class II molecules are formed as two polypeptide chains: alpha and beta. These are typically highly polymorphic from one individual to another, although some haplotypes are much more common in certain populations than others.

Polypeptides for any haplotype or any combination of haplotypes may be used as targets in the present invention including HLA-DRB, HLA-DRB03, HLA-DRB15, HLA-DRB04, HLA-DRB07, HLA-DRB01

HLA-DR has very little polymorphism, making HLA-DRα and/or HLA-DRβ particularly suitable for use as a target in the present invention.

HLA-DP and HLA-DQ have polymorphic α and β chains. Therefore one can select common HLA-DP or HLA-DQ α or β chain and restrict allogeneic production only from recipients with that haplotype. Suitably, the recipient may be homozygous for that haplotype. Wherein the recipient is not homozygous for the haplotype, two HLA-DP and two HLA-DQ (optionally in combination with HLA-DR e.g. HLA-DRα) may be used.

The sequences of MHC polypeptides are provided in the ImMunoGeneTics (IMGT) database (Lefranc, M.-P. et al., Nucleic Acids Res., 27:209-212 (1999); doi: 10.1093/nar/27.1.209).

Suitably, the receptor that downregulates immune response may be selected from programmed cell death protein 1 (PD-1), cytotoxic T-lymphocyte-associated protein 4 (CTLA-4), T-cell immunoglobulin and mucindomain containing-3 (Tim3), killer immunoglobulin-like receptors (KIRs), CD94, NKG2A, TIGIT, BTLA, Fas, TBR2, LAG3 or a protein tyrosine phosphatase.

An exemplary target binding domain which may be used in the present invention is anti-PD1_clone 10 (shown as Ab-3 in FIG. 2) or a variant thereof having at least 80% identity thereto:

Ab-3 (aPD1_clone10):
(SEQ ID NO: 32)
DVLMTQTPLSLPVSLGDQASISCRSGQNIVHSNGNTYLEW
YLQKPGQSPKLLIYKVSNRFFGVPDRISGSGSGTDFTLKI
SRVEAEDLGVYFCFQGSHVPFTFGSGTKLEIKSGGGGSGG
GGSGGGGSDVQLQESGPGLVKPSQSLSLTCTVTGYSITSD
YAWNWIRQFPGNKLEWMGYINYSGSTSYNPSLKSRISITR
DTSKNQFFLQLNSVTTEDTATYYCARWIGSSAWYFDVWGA
GTTVTVSS

Suitably, the cytosolic protein which modulates the immune response may be selected from Csk, SHP1, SHP2, Zap-70, SLP76 and AKT.

Suitably, the ligand expressed on T cells may be CD5, CD7 or CD2.

In one aspect, the engineered immune cell according to the present invention further comprises a chimeric antigen receptor (CAR) or transgenic T cell receptor (TCR).

An exemplary CAR sequence which may be used in the present invention is CD19CAR or a variant thereof having at least 80% identity thereto:

aCD19CAR:
(SEQ ID NO: 33)
DIQMTQTTSSLSASLGDRVTISCRASQDISKYLNWYQQKP
DGTVKLLIYHTSRLHSGVPSRFSGSGSGTDYSLTISNLEQ
EDIATYFCQQGNTLPYTFGGGTKLEITKAGGGGSGGGGSG
GGGSGGGGSEVKLQESGPGLVAPSQSLSVTCTVSGVSLPD
YGVSWIRQPPRKGLEWLGVIWGSETTYYNSALKSRLTIIK
DNSKSQVFLKMNSLQTDDTAIYYCAKHYYYGGSYAMDYWG
QGTSVTVSSDPTTTPAPRPPTPAPTIASQPLSLRPEACRP
AAGGAVHTRGLDFACDIYIWAPLAGTCGVLLLSLVITLYC
KRGRKKLLYIFKQPFMRPVQTTQEEDGCSCRFPEEEEGGC
ELRVKFSRSADAPAYQQGQNQLYNELNLGRREEYDVLDKR
RGRDPEMGGKPRRKNPQEGLYNELQKDKMAEAYSEIGMKG
ERRRGKGHDGLYQGLSTATKDTYDALHMQALPPR

Marker

In one aspect, the one or more nucleic acid construct(s), or the engineered protein, may further comprises at least one marker, preferably said marker is an extracellular binding domain comprising at least one mAb-specific epitope. The epitope may be may be an extracellular domain which is recognised by an antibody.

Markers may be used to measure transduction efficiency, to allow purification of transduced cells and/or facilitate depletion of the engineered cell. Suitably, the marker may be encoded by a suicide gene and facilitate depletion of engineered cells in case of toxicity.

An exemplary marker which may be used in the present invention is RQR8 or a variant thereof having at least 80% identity thereto:

RQR8:
(SEQ ID NO: 34)
CPYSNPSLCSGGGGSELPTQGTFSNVSTNVSPAKPTTTAC
PYSNPSLCSGGGGSPAPRPPTPAPTIASQPLSLRPEACRP
AAGGAVHTRGLDFACDIYIWAPLAGTCGVLLLSLVITLYC
NHRNRRRVCKCPRPVVRA

Rituximab may be used to deplete engineered cells expressing RQR8.

Signal Peptide

The classical protein secretion pathway is through the endoplasmic reticulum (ER). The engineered proteins, markers, CARs and transgenic TCRs described herein may comprise a signal sequence so that when the proteins are expressed inside a cell, the nascent protein is directed to the ER.

The term “signal peptide” is synonymous with “signal sequence”.

A signal peptide is a short peptide, commonly 5-30 amino acids long, typically present at the N-terminus of the majority of newly synthesized proteins that are destined towards the secretory pathway. These proteins include those that reside either inside certain organelles (for example, the endoplasmic reticulum, Golgi or endosomes), are secreted from the cell, and transmembrane proteins.

Signal peptides commonly contain a core sequence which is a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide is commonly positioned at the amino terminus of the molecule, although some carboxy-terminal signal peptides are known.

Signal sequences typically have a tripartite structure, consisting of a hydrophobic core region (h-region) flanked by an n- and c-region. The latter contains the signal peptidase (SPase) consensus cleavage site. Usually, signal sequences are cleaved off co-translationally, the resulting cleaved signal sequences are termed signal peptides.

Signal sequences can be detected or predicted using software techniques (see for example, http://www.predisi.de/).

A very large number of signal sequences are known, and are available in databases. For example, http://www.signalpeptide.de lists 2109 confirmed mammalian signal peptides in its database.

In one embodiment, the protein may be operably linked to a signal peptide which enables translocation of the protein into the endoplasmic reticulum (ER). The protein may be engineered to be operably linked to a signal peptide which enables translocation of the protein into the ER. Suitably, the protein may operably linked to a signal peptide which is not normally operably linked to in nature. Suitably, the combination of the protein and the signal peptide may be synthetic (e.g. not found in nature).

In some embodiments an altered signal peptide (such as a less efficient signal peptide) may be used. The use of an altered signal peptide may allow the system to be tuned according to clinical need. The ratio of proteins may be modified by modulating the efficiency of one or more of the signal peptides on the two proteins. Methods for modulating the efficiency of signal peptides are described in WO2016/174409 (which is incorporated herein by reference).

Suitably, the signal peptide may be a murine Ig kappa chain V-Ill region signal peptide or a variant thereof. The amino acid sequence of a murine Ig kappa chain V-Ill region signal peptide is set forth in SEQ ID NO: 35. Suitably, the signal peptide may comprise the exemplary sequence SEQ ID NO 35 or a variant thereof having at least 80% identity thereto.

(SEQ ID NO: 35)
METDTLILWVLLLLVPGSTG

Suitably, the signal peptide may comprise a sequence set forth in the exemplary sequence SEQ ID NO: 36 or a variant thereof having at least 80% identity thereto.

(SEQ ID NO: 36)
MGTSLLCWMALCLLGADHADA

Variant sequences may have at least 80%, 85%, 90%, 95%, 98% or 99% sequence identity to SEQ ID NO: 35-36, provided that the sequence is able to function as a signal peptide. The variant sequence retains the ability to direct the nascent protein to the ER.

Chimeric Antigen Receptor (Car)

Classical CARs are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ (results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 4-1 BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a wild-type ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following table. The antigen-binding domain used in the present invention may be a domain which is capable of binding a TAA as indicated therein.

TABLE 6

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Transmembrane Domain

The transmembrane domain is the sequence of a classical CAR that spans the membrane. It may comprise a hydrophobic alpha helix. The transmembrane domain may be derived from CD28, which gives good receptor stability.

CAR or TCR Signal Peptide

The CAR or transgenic TCR for use in the present invention may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Spacer Domain

The receptor may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together.

The intracellular signalling domain may be or comprise a T cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino acids, giving the signature YxxL/1. Two of these signatures are typically separated by between 6 and 8 amino acids in the tail of the molecule (YxxL/Ix₍₆₋₈₎YxxL/I).

ITAMs are important for signal transduction in immune cells. Hence, they are found in the tails of important cell signalling molecules such as the CD3 and ζ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

The intracellular signalling domain component may comprise, consist essentially of, or consist of the CD3-ζ (endodomain, which contains three ITAMs. Classically, the CD3-ζ (endodomain transmits an activation signal to the T cell after antigen is bound.

The intracellular signalling domain may comprise additional co-stimulatory signalling. For example, 4-1BB (also known as CD137) can be used with CD3-ζ, or CD28 and OX40 can be used with CD3-ζ to transmit a proliferative/survival signal.

Suitably, the CAR may have the general format: antigen-binding domain-TCR element.

As used herein “TCR element” means a domain or portion thereof of a component of the TCR receptor complex. The TCR element may comprise (e.g. have) an extracellular domain and/or a transmembrane domain and/or an intracellular domain e.g. intracellular signalling domain of a TCR element.

The TCR element may selected from TCR alpha chain, TCR beta chain, a CD3 epsilon chain, a CD3 gamma chain, a CD3 delta chain, CD3 epsilon chain.

Suitably, the TCR element may comprise the extracellular domain of the TCR alpha chain, TCR beta chain, a CD3 epsilon chain, a CD3 gamma chain, a CD3 delta chain, or CD3 epsilon chain. Suitably, the TCR element may comprise the transmembrane domain of the TCR alpha chain, TCR beta chain, a CD3 epsilon chain, a CD3 gamma chain, a CD3 delta chain, or CD3 epsilon chain. Suitably, the TCR element may comprise the intracellular domain of the TCR alpha chain, TCR beta chain, a CD3 epsilon chain, a CD3 gamma chain, a CD3 delta chain, or CD3 epsilon chain. Suitably, the TCR element may comprise the TCR alpha chain, TCR beta chain, a CD3 epsilon chain, a CD3 gamma chain, a CD3 delta chain, or CD3 epsilon chain.

Transgenic T-Cell Receptor (TCR)

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example the genes for engineered TCRs may be reintroduced into autologous T cells and transferred back into patients for T cell adoptive therapies. Such ‘heterologous’ TCRs may also be referred to herein as ‘transgenic TCRs’.

Nucleic Acid Construct/Kit of Nucleic Acid Sequences

The present invention provides one or more nucleic acid constructs which together encode at least two target-binding domains, wherein the one or more nucleic acid constructs together contain a single nucleotide sequence encoding an intracellular retention signal which, when co-expressed in the cell, controls the cellular localisation of each of the target-binding domains.

Suitably, one or more may refer to one, two, three or four nucleic acid constructs.

Suitably, the present invention provides one or two nucleic acids constructs which together encoder the elements according to the present invention. Minimising the total number of nucleic acid constructs required to provide the elements of the invention reduces the disadvantages associated with requiring multiple constructs to be introduced into a target cell.

In one aspect, each of the at least two target-binding domains and the intracellular retention signal are encoded as separate polypeptides; each of the polypeptides comprising the target-binding domains further comprises a first protein interaction domain and the polypeptide comprising the intracellular retention signal further comprises a second protein interaction domain wherein the first and second protein interaction domain are capable of binding to each other.

In another aspect, the present invention provides a nucleic acid construct which comprises the following structure:

A-X-B-C
in which:
A and B are nucleic acid sequences encoding a target-binding domain as defined herein; X is a linker as defined herein; and C is an intracellular retention signal as defined herein.

Suitably, the nucleic acid construct may further comprise one or more additional nucleic acid sequences encoding an additional target-binding domain(s). The additional target-binding domains are preferably coupled to the intracellular retention signal.

The nuclic acid construct may further comprise a nucleic acid sequence which encodes a CAR or a transgenic TCR or at least one marker, such as an extracellular binding domain. An exemplary marker is RQR8 or a variant thereof.

As used herein, the terms “polynucleotide”, “nucleotide”, and “nucleic acid” are intended to be synonymous with each other.

Suitably, the nucleic acid construct may comprise a plurality of nucleic acid sequences which encode components of the invention such as at least two target-binding proteins and an intracellular retention signal, optionally further comprising additional target-binding domains, a CAR, a transgenic TCR, a marker. For example, the nucleic acid construct may comprise two, three, four or more nucleic acid sequences which encode different components of the invention. Suitably, the plurality of nucleic acid sequences may be separated by co-expression sites as defined herein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed. Suitably, the polynucleotides of the present invention are codon optimised to enable expression in a mammalian cell, in particular a cytolytic immune cell as described herein.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Co-Expression Site

A co-expression site is used herein to refer to a nucleic acid sequence enabling co-expression of nucleic acid sequences encoding target-binding proteins and other engineered components of the engineered immune cell according to the present invention such as: CARs, transgenic TCRs and heteromultimeric polypeptide components.

Suitably, there may be a co-expression site between a first nucleic acid sequence and a second nucleic acid sequence. Suitably, in embodiments where a plurality of co-expression sites is present in the engineered polynucleotide, the same co-expression site may be used.

Preferably, the co-expression site is a cleavage site. The cleavage site may be any sequence which enables the two polypeptides to become separated. The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide. A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above.

Exemplary 2A sequences which may be used in the present invention include:

EGRGSLLTCGDVEENPGP (SEQ ID NO: 37) or a variant thereof having at least 80% sequence identity to SEQ ID NO: 37 and which retains the ability to function as a cleavage site.

The co-expression sequence may be an internal ribosome entry sequence (IRES). The co-expressing sequence may be an internal promoter.

Vector

The present invention also provides a vector, which comprises one or more nucleic acid sequence(s) or nucleic acid construct(s) of the invention. Such a vector may be used to introduce the nucleic acid sequence(s) or construct(s) into a host cell so that it expresses an engineered protein which comprises at least two target-binding domains coupled to an intracellular retention signal as defined herein.

Suitably, the vector may comprise a plurality of nucleic acid sequences which encode different components as provided by the present invention. For example, the vector may comprise two, three, four or more nucleic acid sequences which encode different components of the invention, such as the at least two target-binding domains, an intracellular retention signal and a marker, a CAR or transgenic TCR. Suitably, the plurality of nucleic acid sequences may be separated by co-expression sites as defined herein.

The vector may, for example, be a plasmid or a viral vector, such as a retroviral vector or a lentiviral vector, or a transposon based vector or synthetic mRNA.

The vector may be capable of transfecting or transducing a cell.

Pharmaceutical Composition

The present invention also relates to a pharmaceutical composition comprising an engineered immune cell according to the present invention or a cell obtainable (e.g. obtained) by a method according to the present invention.

The present invention also provides a pharmaceutical composition comprising, a nucleic acid construct according to the present invention, a group of nucleic acid sequences as defined herein or a vector according to the present invention. In particular, the invention relates to a pharmaceutical composition containing a cell according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering an engineered immune cell according to the invention, or obtainable (e.g. obtained) by a method according to the present invention, or a nucleic acid construct according to the present invention, or a group of nucleic acid sequences as defined herein; or a vector according to the present invention (for example in a pharmaceutical composition as described above) to a subject.

Suitably, the present methods for treating and/or preventing a disease may comprise administering an engineered immune cell according to the present invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolation of a cell-containing sample;
(ii) introduction of the nucleic acid construct according to the present invention, a group of nucleic acid sequence as defined herein, or a vector according to the present invention to the cell; and
(iii) administering the cells from (ii) to a subject.

The method may involve the steps of:
(i) introduction of the nucleic acid construct according to the present invention, a group of nucleic acid sequence as defined herein, or a vector according to the present invention to a cell; and
(iii) administering the cells from (ii) to a subject.

Suitably, the nucleic acid construct, vector(s) or nucleic acids may be introduced by transduction. Suitably, the nucleic acid construct, vector(s) or nucleic acids may be introduced by transfection.

Suitably, the cell may be autologous. Suitably, the cell may be allogenic.

The present invention provides an engineered immune cell according to the present invention, a nucleic acid construct according to the present invention, a group of nucleic acid sequences as defined herein, or a vector according to the present invention, for use in treating and/or preventing a disease. In particular the present invention provides an engineered immune cell of the present invention for use in treating and/or preventing a disease.

The present invention also relates to an engineered immune cell according to the present invention, a nucleic acid construct according to the present invention, a group of nucleic acid sequences as defined herein, or a vector according to the present invention, in the manufacture of a medicament for the treatment and/or prevention of a disease. In particular, the invention relates to the use of an engineered immune cell according to the present invention in the manufacture of a medicament for the treatment and/or prevention of a disease.

The disease to be treated and/or prevented by the method of the present invention may be cancer.

The cancer may be a cancer such as neuroblastoma, prostate cancer, bladder cancer, breast cancer, colon cancer, endometrial cancer, kidney cancer (renal cell), leukaemia, lung cancer, melanoma, non-Hodgkin lymphoma, pancreatic cancer, and thyroid cancer.

The cell of the present invention may be capable of killing target cells, such as cancer cells. The target cell may be recognisable by expression of a TAA, for example the expression of a TAA listed in the table above.

Method of Making a Cell

Engineered immune cells of the present invention may be generated by introducing DNA or RNA coding for the engineered protein which comprises at least two target-binding domains coupled to an intracellular retention signal as defined herein by one of many means including transduction with a viral vector, transfection with DNA or RNA.

The cell of the invention may be made by introducing to a cell (e.g. by transduction or transfection) the nucleic acid construct or vector according to the present invention, or a group of nucleic acid sequences as defined above, or a vector according to the present invention.

Suitably, the cell may be from a sample isolated from a subject.

As used herein, the term “introduced” or “introducing” refer to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector.

Engineered cells according to the present invention may be generated by introducing DNA or RNA coding for the releasable protein and the retention protein by one of many means including transduction with a viral vector, transfection with DNA or RNA.

Cells may be activated and/or expanded prior to the introduction of a nucleic acid sequence, for example by treatment with an anti-CD3 monoclonal antibody or both anti-CD3 and anti-CD28 monoclonal antibodies. As used herein “activated” means that a cell has been stimulated, causing the cell to proliferate, differentiate or initiate an effector function.

Methods for measuring cell activation are known in the art and include, for example, measuring the expression of activation markers by flow cytometry, such as the expression of CD69, CD25, CD38 or HLA-DR or measuring intracellular cytokines.

As used herein “expanded” means that a cell or population of cells has been induced to proliferate.

The expansion of a population of cells may be measured for example by counting the number of cells present in a population. The phenotype of the cells may be determined by methods known in the art such as flow cytometry.

The illustrative nucleic acid constructs described in the figures encode the following polyproteins which comprise the various components in the order they are listed. The one ore more nucleic acid constructs of the invention may encode a polyprotein(s) as shown in figures; or variants thereof as described herein.

FIG. 2 construct amino acid
sequence from N to C-terminus
Signal sequence:
(SEQ ID NO: 36)
MGTSLLCWMALCLLGADHADA
RQR8:
(SEQ ID NO: 34)
CPYSNPSLCSGGGGSELPTQGTFSNVSTNVSPAKPTTTACPYSNP
SLCSGGGGSPAPRPPTPAPTIASQPLSLRPEACRPAAGGAVHTRG
LDFACDIYIWAPLAGTCGVLLLSLVITLYCNHRNRRRVCKCPRPV
VRA
2A:
(SEQ ID NO: 37)
EGRGSLLTCGDVEENPGP
Signal sequence:
(SEQ ID NO: 35)
METDTLILWVLLLLVPGSTG
Ab-1 (aCD3e_UCHT):
(SEQ ID NO: 30)
DIQMTQSPSSLSASVGNRVTITCRASQDIRNYLNWYQQKPGKAPK
LLIYYTSRLESGVPSRFSGSGSGTDYTLTISSLQPEDFATYYCQQ
GNTLPWTFGQGTKVEIKSGGGGSGGGGSGGGGSEVQLVESGGGLV
QPGGSLRLSCAASGYSFTGYTMNWVRQAPGKGLEWVALINPYKGV
STYNQKFKDRFTISVDKSKNTAYLQMNSLRAEDTAVYYCARSGYY
GDSDWYFDVWGQGTLVTVSS
Linker (L1):
(SEQ ID NO: 11)
SGGGSGGGSGGGS
Ab-2 (aB2M_dN6B2m):
(SEQ ID NO: 31)
QVQLQESGGGSVQAGGSLRLSCAASGYTDSRYCMAWFRQAPGKER
EWVARINSGRDITYYADSVKGRFTFSQDNAKNTVYLQMDSLEPED
TATYYCATDIPLRCRDIVAKGGDGFRYWGQGTQVTVSS
Linker (L2):
(SEQ ID NO: 11)
SGGGSGGGSGGGS
Ab-3 (aPD1_clone10):
(SEQ ID NO: 32)
DVLMTQTPLSLPVSLGDQASISCRSGQNIVHSNGNTYLEWYLQKP
GQSPKLLIYKVSNRFFGVPDRISGSGSGTDFTLKISRVEAEDLGV
YFCFQGSHVPFTFGSGTKLEIKSGGGGSGGGGSGGGGSDVQLQES
GPGLVKPSQSLSLTCTVTGYSITSDYAWNWIRQFPGNKLEWMGYI
NYSGSTSYNPSLKSRISITRDTSKNQFFLQLNSVTTEDTATYYCA
RWIGSSAWYFDVWGAGTTVTVSS
KDEL:
(SEQ ID NO: 1)
SEKDEL
FIG. 3a construct amino acid sequence from
N to C-terminus
Signal sequence:
(SEQ ID NO: 36)
MGTSLLCWMALCLLGADHADA
RQR8:
(SEQ ID NO: 34)
CPYSNPSLCSGGGGSELPTQGTFSNVSTNVSPAKPTTTACPYSNP
SLCSGGGGSPAPRPPTPAPTIASQPLSLRPEACRPAAGGAVHTRG
LDFACDIYIWAPLAGTCGVLLLSLVITLYCNHRNRRRVCKCPRPV
VRA
2A:
(SEQ ID NO: 37)
EGRGSLLTCGDVEENPGP
Signal sequence:
(SEQ ID NO: 35)
METDTLILWVLLLLVPGSTG
aCD19CAR:
(SEQ ID NO: 38)
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLEITKAGGGGSGGGGSGGGGSGGGGSEVKLQE
SGPGLVAPSQSLSVTCTVSGVSLPDYGVSWIRQPPRKGLEWLGVI
WGSETTYYNSALKSRLTIIKDNSKSQVFLKMNSLQTDDTAIYYCA
KHYYYGGSYAMDYWGQGTSVTVSSDPTTTPAPRPPTPAPTIASQP
LSLRPEACRPAAGGAVHTRGLDFACDIYIWAPLAGTCGVLLLSLV
ITLYCKRGRKKLLYIFKQPFMRPVQTTQEEDGCSCRFPEEEEGGC
ELRVKFSRSADAPAYQQGQNQLYNELNLGRREEYDVLDKRRGRDP
EMGGKPRRKNPQEGLYNELQKDKMAEAYSEIGMKGERRRGKGHDG
LYQGLSTATKDTYDALHMQALPPR
2A:
(SEQ ID NO: 37)
EGRGSLLTCGDVEENPGP
Signal sequence:
(SEQ ID NO: 35)
METDTLILWVLLLLVPGSTG
Ab-1 (aCD3e_UCHT):
(SEQ ID NO: 30)
DIQMTQSPSSLSASVGNRVTITCRASQDIRNYLNWYQQKPGKAPK
LLIYYTSRLESGVPSRFSGSGSGTDYTLTISSLQPEDFATYYCQQ
GNTLPWTFGQGTKVEIKSGGGGSGGGGSGGGGSEVQLVESGGGLV
QPGGSLRLSCAASGYSFTGYTMNWVRQAPGKGLEWVALINPYKGV
STYNQKFKDRFTISVDKSKNTAYLQMNSLRAEDTAVYYCARSGYY
GDSDWYFDVWGQGTLVTVSS
Kappa C:
(SEQ ID NO: 14)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
Ab-2 (aB2M_dN6B2m):
(SEQ ID NO: 31)
QVQLQESGGGSVQAGGSLRLSCAASGYTDSRYCMAWFRQAPGKER
EWVARINSGRDITYYADSVKGRFTFSQDNAKNTVYLQMDSLEPED
TATYYCATDIPLRCRDIVAKGGDGFRYWGQGTQVTVSS
2A:
(SEQ ID NO: 37)
EGRGSLLTCGDVEENPGP
Signal sequence:
(SEQ ID NO: 35)
METDTLILWVLLLLVPGSTG
Ab-3 (aPD1_clone10):
(SEQ ID NO: 32)
DVLMTQTPLSLPVSLGDQASISCRSGQNIVHSNGNTYLEWYLQKP
GQSPKLLIYKVSNRFFGVPDRISGSGSGTDFTLKISRVEAEDLGV
YFCFQGSHVPFTFGSGTKLEIKSGGGGSGGGGSGGGGSDVQLQES
GPGLVKPSQSLSLTCTVTGYSITSDYAWNWIRQFPGNKLEWMGYI
NYSGSTSYNPSLKSRISITRDTSKNQFFLQLNSVTTEDTATYYCA
RWIGSSAWYFDVWGAGTTVTVSS
CH1:
(SEQ ID NO: 15)
STKGPSVFPLAPSSKSTSGGTAALGCLVKDYFPEPVTVSWNSGAL
TSGVHTFPAVLQSSGLYSLSSVVTVPSSSLGTQTYICNVNHKPSN
TKVDKRV
KDEL:
(SEQ ID NO: 1)
SEKDEL
FIG. 3b construct amino acid
sequence from N to C-terminus
Plasmid 1
Signal sequence:
(SEQ ID NO: 36)
MGTSLLCWMALCLLGADHADA
RQR8:
(SEQ ID NO: 34)
CPYSNPSLCSGGGGSELPTQGTFSNVSTNVSPAKPTTTACPYSNP
SLCSGGGGSPAPRPPTPAPTIASQPLSLRPEACRPAAGGAVHTRG
LDFACDIYIWAPLAGTCGVLLLSLVITLYCNHRNRRRVCKCPRPV
VRA
2A:
(SEQ ID NO: 37)
EGRGSLLTCGDVEENPGP
Signal sequence:
(SEQ ID NO: 35)
METDTLILWVLLLLVPGSTG
Ab-1 (aCD3e_UCHT):
(SEQ ID NO: 30)
DIQMTQSPSSLSASVGNRVTITCRASQDIRNYLNWYQQKPGKAPK
LLIYYTSRLESGVPSRFSGSGSGTDYTLTISSLQPEDFATYYCQQ
GNTLPWTFGQGTKVEIKSGGGGSGGGGSGGGGSEVQLVESGGGLV
QPGGSLRLSCAASGYSFTGYTMNWVRQAPGKGLEWVALINPYKGV
STYNQKFKDRFTISVDKSKNTAYLQMNSLRAEDTAVYYCARSGYY
GDSDWYFDVWGQGTLVTVSS
Kappa C:
(SEQ ID NO: 14)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
Ab-2 (aB2M_dN6B2m):
(SEQ ID NO: 31)
QVQLQESGGGSVQAGGSLRLSCAASGYTDSRYCMAWFRQAPGKER
EWVARINSGRDITYYADSVKGRFTFSQDNAKNTVYLQMDSLEPED
TATYYCATDIPLRCRDIVAKGGDGFRYWGQGTQVTVSS
Plasmid 2
Signal sequence:
(SEQ ID NO: 35)
METDTLILWVLLLLVPGSTG
aCD19CAR:
(SEQ ID NO: 38)
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLEITKAGGGGSGGGGSGGGGSGGGGSEVKLQE
SGPGLVAPSQSLSVTCTVSGVSLPDYGVSWIRQPPRKGLEWLGVI
WGSETTYYNSALKSRLTIIKDNSKSQVFLKMNSLQTDDTAIYYCA
KHYYYGGSYAMDYWGQGTSVTVSSDPTTTPAPRPPTPAPTIASQP
LSLRPEACRPAAGGAVHTRGLDFACDIYIWAPLAGTCGVLLLSLV
ITLYCKRGRKKLLYIFKQPFMRPVQTTQEEDGCSCRFPEEEEGGC
ELRVKFSRSADAPAYQQGQNQLYNELNLGRREEYDVLDKRRGRDP
EMGGKPRRKNPQEGLYNELQKDKMAEAYSEIGMKGERRRGKGHDG
LYQGLSTATKDTYDALHMQALPPR
2A:
(SEQ ID NO: 37)
EGRGSLLTCGDVEENPGP
Signal sequence:
(SEQ ID NO: 35)
METDTLILWVLLLLVPGSTG
Ab-3 (aPD1_clone10):
(SEQ ID NO: 32)
DVLMTQTPLSLPVSLGDQASISCRSGQNIVHSNGNTYLEWYLQKP
GQSPKLLIYKVSNRFFGVPDRISGSGSGTDFTLKISRVEAEDLGV
YFCFQGSHVPFTFGSGTKLEIKSGGGGSGGGGSGGGGSDVQLQES
GPGLVKPSQSLSLTCTVTGYSITSDYAWNWIRQFPGNKLEWMGYI
NYSGSTSYNPSLKSRISITRDTSKNQFFLQLNSVTTEDTATYYCA
RWIGSSAWYFDVWGAGTTVTVSS
CH1:
(SEQ ID NO: 15)
STKGPSVFPLAPSSKSTSGGTAALGCLVKDYFPEPVTVSWNSGAL
TSGVHTFPAVLQSSGLYSLSSVVTVPSSSLGTQTYICNVNHKPSN
TKVDKRV
KDEL:
(SEQ ID NO: 1)
SEKDEL
FIG. 3b construct amino acid
sequence from N to C-terminus
Signal sequence:
(SEQ ID NO: 36)
MGTSLLCWMALCLLGADHADA
RQR8:
(SEQ ID NO: 34)
CPYSNPSLCSGGGGSELPTQGTFSNVSTNVSPAKPTTTACPYSNP
SLCSGGGGSPAPRPPTPAPTIASQPLSLRPEACRPAAGGAVHTRG
LDFACDIYIWAPLAGTCGVLLLSLVITLYCNHRNRRRVCKCPRPV
VRA
2A:
(SEQ ID NO: 37)
EGRGSLLTCGDVEENPGP
Signal sequence:
(SEQ ID NO: 35)
METDTLILWVLLLLVPGSTG
Ab-1 (aCD3e_UCHT):
(SEQ ID NO: 30)
DIQMTQSPSSLSASVGNRVTITCRASQDIRNYLNWYQQKPGKAPK
LLIYYTSRLESGVPSRFSGSGSGTDYTLTISSLQPEDFATYYCQQ
GNTLPWTFGQGTKVEIKSGGGGSGGGGSGGGGSEVQLVESGGGLV
QPGGSLRLSCAASGYSFTGYTMNWVRQAPGKGLEWVALINPYKGV
STYNQKFKDRFTISVDKSKNTAYLQMNSLRAEDTAVYYCARSGYY
GDSDWYFDVWGQGTLVTVSS
Kappa C:
(SEQ ID NO: 14)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
Ab-2 (aB2M_dN6B2m):
(SEQ ID NO: 31)
QVQLQESGGGSVQAGGSLRLSCAASGYTDSRYCMAWFRQAPGKER
EWVARINSGRDITYYADSVKGRFTFSQDNAKNTVYLQMDSLEPED
TATYYCATDIPLRCRDIVAKGGDGFRYWGQGTQVTVSS
2A:
(SEQ ID NO:37)
EGRGSLLTCGDVEENPGP
Signal sequence:
(SEQ ID NO: 35)
METDTLILWVLLLLVPGSTG
Ab-3 (aPD1_clone10):
(SEQ ID NO: 32)
DVLMTQTPLSLPVSLGDQASISCRSGQNIVHSNGNTYLEWYLQKP
GQSPKLLIYKVSNRFFGVPDRISGSGSGTDFTLKISRVEAEDLGV
YFCFQGSHVPFTFGSGTKLEIKSGGGGGGGGSGGGGSDVQLQESG
PGLVKPSQSLSLTCTVTGYSITSDYAWNWIRQFPGNKLEWMGYIN
YSGSTSYNPSLKSRISITRDTSKNQFFLQLNSVTTEDTATYYCAR
WIGSSAWYFDVWGAGTTVTVSS
CD79a:
(SEQ ID NO: 12)
LWMHKVPASLMVSLGEDAHFQCPHNSSNNANVTWWRVLHGNYTWP
PEFLGPGEDPNGTLIIQNVNKSHGGIYVCRVQEGNESYQQSCGTY
LRVRQPPPRPFLDMGEGTKNR
Ab-4 (aCD52_):
(SEQ ID NO: 39)
DIQMTQSPSSLSASVGDRVTITCKASQNIDKYLNWYQQKPGKAPK
LLIYNTNNLQTGVPSRFSGSGSGTDFTFTISSLQPEDIATYYCLQ
HISRPRTFGQGTKVEIKSGGGGSGGGGSGGGGSQVQLQESGPGLV
RPSQTLSLTCTVSGFTFTDFYMNWVRQPPGRGLEWIGFIRDKAKG
YTTEYNPSVKGRVTMLVDTSKNQFSLRLSSVTAADTAVYYCAREG
HTAAPFDYWGQGSLVTVSS
2A:
(SEQ ID NO: 37)
EGRGSLLTCGDVEENPGP
Signal sequence:
(SEQ ID NO: 35)
METDTLILWVLLLLVPGSTG
Ab-5 (aTBR2_E11):
(SEQ ID NO: 40)
QVQLQESGGGLVQPGGSLRLSCAASGIILSSKAVAWYRQPPGQQR
EGVAHSSVSGTTIYADSVKGRFTVSRDNAKNTVYLEMNSLKPEDT
AVYYCTAPVGHWGQGTQVTVSS
CD79b:
(SEQ ID NO: 13)
ARSEDRYRNPKGSACSRIWQSPRFIARKRGFTVKMHCYMNSASGN
VSWLWKQEMDENPQQLKLEKGRMEESQNESLATLTIQGIRFEDNG
IYFCQQKCNNTSEVYQGCGTELRVMGFSTLAQLKQRNTLKD
Linker:
(SEQ ID NO: 11)
SGGGSGGGSGGGS
CH1:
(SEQ ID NO: 15)
STKGPSVFPLAPSSKSTSGGTAALGCLVKDYFPEPVTVSWNSGAL
TSGVHTFPAVLQSSGLYSLSSVVTVPSSSLGTQTYICNVNHKPSN
TKVDKRV
KDEL:
(SEQ ID NO: 1)
SEKDEL
FIG. 4 construct amino acid
sequence from N to C-terminus
Signal sequence:
(SEQ ID NO: 36)
MGTSLLCWMALCLLGADHADA
RQR8:
(SEQ ID NO: 34)
CPYSNPSLCSGGGGSELPTQGTFSNVSTNVSPAKPTTTACPYSNP
SLCSGGGGSPAPRPPTPAPTIASQPLSLRPEACRPAAGGAVHTRG
LDFACDIYIWAPLAGTCGVLLLSLVITLYQNHRNRRRVCKCPRPV
VRA
2A:
(SEQ ID NO: 37)
EGRGSLLTCGDVEENPGP
Signal sequence:
(SEQ ID NO: 35)
METDTLILWVLLLLVPGSTG
Ab-1 (aCD3e_UCHT):
(SEQ ID NO: 30)
DIQMTQSPSSLSASVGNRVTITCRASQDIRNYLNWYQQKPGKAPK
LLIYYTSRLESGVPSRFSGSGSGTDYTLTISSLQPEDFATYYCQQ
GNTLPWTFGQGTKVEIKSGGGGSGGGGSGGGGSEVQLVESGGGLV
QPGGSLRLSCAASGYSFTGYTMNWVRQAPGKGLEWVALINPYKGV
STYNQKFKDRFTISVDKSKNTAYLQMNSLRAEDTAVYYCARSGYY
GDSDWYFDVWGQGTLVTVSS
ALFA_tag:
(SEQ ID NO: 21)
PSRLEEELRRRLTEP
2A:
(SEQ ID NO: 37)
EGRGSLLTCGDVEENPGP
Signal sequence:
(SEQ ID NO: 35)
METDTLILWVLLLLVPGSTG
Ab-2 (aB2M_dN6B2m):
(SEQ ID NO: 31)
QVQLQESGGGSVQAGGSLRLSCAASGYTDSRYCMAWFRQAPGKER
EWVARINSGRDITYYADSVKGRFTFSQDNAKNTVYLQMDSLEPED
TATYYCATDIPLRCRDIVAKGGDGFRYWGQGTQVTVSS
ALFA_tag:
(SEQ ID NO: 21)
PSRLEEELRRRLTEP
2A:
(SEQ ID NO: 37)
EGRGSLLTCGDVEENPGP
Signal sequence:
(SEQ ID NO: 35)
METDTLILWVLLLLVPGSTG
Ab-3 (aPD1_clone10):
(SEQ ID NO: 32)
DVLMTQTPLSLPVSLGDQASISCRSGQNIVHSNGNTYLEWYLQKP
GQSPKLLIYKVSNRFFGVPDRISGSGSGTDFTLKISRVEAEDLGV
YFCFQGSHVPFTFGSGTKLEIKSGGGGSGGGGSGGGGSDVQLQES
GPGLVKPSQSLSLTCTVTGYSITSDYAWNWIRQFPGNKLEWMGYI
NYSGSTSYNPSLKSRISITRDTSKNQFFLQLNSVTTEDTATYYCA
RWIGSSAWYFDVWGAGTTVTVSS
ALFA_tag:
(SEQ ID NO: 21)
PSRLEEELRRRLTEP
2A:
(SEQ ID NO: 37)
EGRGSLLTCGDVEENPGP
Signal sequence:
(SEQ ID NO: 35)
METDTLILWVLLLLVPGSTG
Anti-ALFA_Tag (NbALFA):
(SEQ ID NO: 22)
EVQLQESGGGLVQPGGSLRLSCTASGVTISALNAMAMGWYRQAPG
ERRVMVAAVSERGNAMYRESVQGRFTVTRDFTNKMVSLQMDNLKP
EDTAVYYCHVLEDRVDSFHDYWGQGTQVTVSS
KDEL:
(SEQ ID NO: 1)
SEKDEL
FIG. 5 construct amino acid
sequence from N to C-terminus
Signal sequence:
(SEQ ID NO: 36)
MGTSLLCWMALCLLGADHADA
RQR8:
(SEQ ID NO: 34)
CPYSNPSLCSGGGGSELPTQGTFSNVSTNVSPAKPTTTACPYSNP
SLCSGGGGSPAPRPPTPAPTIASQPLSLRPEACRPAAGGAVHTRG
LDFACDIYIWAPLAGTCGVLLLSLVITLYCNHRNRRRVCKCPRPV
VRA
2A:
(SEQ ID NO: 37)
EGRGSLLTCGDVEENPGP
Signal sequence:
(SEQ ID NO: 35)
METDTLILWVLLLLVPGSTG
aCD19CAR:
(SEQ ID NO: 38)
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLEITKAGGGGSGGGGSGGGGSGGGGSEVKLQE
SGPGLVAPSQSLSVTCTVSGVSLPDYGVSWIRQPPRKGLEWLGVI
WGSETTYYNSALKSRLTIIKDNSKSQVFLKMNSLQTDDTAIYYCA
KHYYYGGSYAMDYWGQGTSVTVSSDPTTTPAPRPPTPAPTIASQP
LSLRPEACRPAAGGAVHTRGLDFACDIYIWAPLAGTCGVLLLSLV
ITLYCKRGRKKLLYIFKQPFMRPVQTTQEEDGCSCRFPEEEEGGC
ELRVKFSRSADAPAYQQGQNQLYNELNLGRREEYDVLDKRRGRDP
EMGGKPRRKNPQEGLYNELQKDKMAEAYSEIGMKGERRRGKGHDG
LYQGLSTATKDTYDALHMQALPPR
2A:
(SEQ ID NO: 37)
EGRGSLLTCGDVEENPGP
Signal sequence:
(SEQ ID NO: 35)
METDTLILWVLLLLVPGSTG
Ab-1 (aCD3e_UCHT):
(SEQ ID NO: 30)
DIQMTQSPSSLSASVGNRVTITCRASQDIRNYLNWYQQKPGKAPK
LLIYYTSRLESGVPSRFSGSGSGTDYTLTISSLQPEDFATYYCQQ
GNTLPWTFGQGTKVEIKSGGGGSGGGGSGGGGSEVQLVESGGGLV
QPGGSLRLSCAASGYSFTGYTMNWVRQAPGKGLEWVALINPYKGV
STYNQKFKDRFTISVDKSKNTAYLQMNSLRAEDTAVYYCARSGYY
GDSDWYFDVWGQGTLVTVSS
ALFA_tag:
(SEQ ID NO: 21)
PSRLEEELRRRLTEP
2A:
(SEQ ID NO: 37)
EGRGSLLTCGDVEENPGP
Ab-2 (aB2M_dN6B2m):
(SEQ ID NO: 31)
QVQLQESGGGSVQAGGSLRLSCAASGYTDSRYCMAWFRQAPGKER
EWVARINSGRDITYYADSVKGRFTFSQDNAKNTVYLQMDSLEPED
TATYYCATDIPLRCRDIVAKGGDGFRYWGQGTQVTVSS
ALFA_tag:
(SEQ ID NO: 21)
PSRLEEELRRRLTEP
2A:
(SEQ ID NO: 37)
EGRGSLLTCGDVEENPGP
Signal sequence:
(SEQ ID NO: 35)
METDTLILWVLLLLVPGSTG
Anti-ALFA_Tag (NbALFA):
(SEQ ID NO: 22)
EVQLQESGGGLVQPGGSLRLSCTASGVTISALNAMAMGWYRQAPG
ERRVMVAAVSERGNAMYRESVQGRFTVTRDFTNKMVSLQMDNLKP
EDTAVYYCHVLEDRVDSFHDYWGQGTQVTVSS
CD8STK:
(SEQ ID NO: 18)
PTTTPAPRPPTPAPTIASQPLSLRPEACRPAAGGAVHTRGLDFACDI
Tyrp-TM:
(SEQ ID NO: 16)
IIAIAVVGALLLVALIFGTASYLI
Linker:
(SEQ ID NO: 11)
SGGGSGGGSGGGS
Anti-ALFA_Tag (NbALFA):
(SEQ ID NO: 22)
EVQLQESGGGLVQPGGSLRLSCTASGVTISALNAMAMGWYRQAPG
ERRVMVAAVSERGNAMYRESVQGRFTVTRDFTNKMVSLQMDNLKP
EDTAVYYCHVLEDRVDSFHDYWGQGTQVTVSS
dCD20_N-terminal:
(SEQ ID NO: 19)
TTPRNSVNGTFPAEPMKGPIAMQSGPKPLFRRMSSLVGPTQSFFM
RESKTLGAVQ
dCD20_TM:
(SEQ ID NO: 17)
IMNGLFHIALGGLLMIPAGIYA
dCD20_Short_Loop:
(SEQ ID NO: 20)
PICVTV
KDEL:
(SEQ ID NO: 1)
SEKDEL

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5 to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

The invention will now be further described by way of Examples, which are meant to serve to assist one of ordinary skill in the art in carrying out the invention and are not intended in any way to limit the scope of the invention.

### Examples

### Example 1—“Daisy-Chain” Linked Binders

Target-binding domains directed against CD3e, B2M and PD1 are sequentially linked followed by a KDEL sequence at the C-terminus. The construct comprises an RQR8 marker followed by a 2A self-cleaving peptide, followed by the daisy chained binders with the KDEL sequence on the C-terminus (see FIG. 2). The construct is transduced into PD1-positive Jurkats and activated PBMCs and the surface expression levels of TCR, HLA and PD1 is assessed by flow cytometry.

### Example 2—Heteromultmeric Coupled Protein

Target-binding domains directed against CD3e and B2M are provided on a kappa domain containing polypeptide chain and a further target-binding domain to PD1 is provided on a second CH1 domain containing polypeptide chain followed by the KDEL sequence at the C-terminus. These two polypeptide chains are either encoded on the same plasmid separated by a 2A peptide (see FIG. 3a) or encoded on two separate plasmids and used in a double transduction (see FIG. 3b). In other embodiments two additional binders can be added with the addition of a CD79 heterodimer (see FIG. 3c). These constructs are transduced into PD1 positive Jurkats and activated PBMCs and the surface expression levels of TCR, HLA and PD1 is assessed by flow cytometry.

### Example 3—“Peptide-Tag” Linked Binders

Target-binding domains directed against CD3e, B2M and PD1 are tagged with an ALFA peptide (small alpha helical peptide structure) on either on the N- or C-terminus and separated by 2A self-cleaving peptides. The final polypeptide chain comprises an anti-ALFA Dab tag-binding protein (NbALFA) followed by the KDEL sequence at the C-terminus (see FIG. 4). This construct is transduced into PD1 positive Jurkats and activated PBMCs and the surface expression levels of TCR, HLA and PD1 is assessed by flow cytometry.

### Example 4—Linked Binders Against an Extracellular and an Intracellular Target Protein

Target-binding domains directed against B2M and SHP2 are tagged with the ALFA peptide and separated by 2A self-cleaving peptides (the SHP2 tagged binder will not contain a signal sequence to ensure cytosolic localisation). The final polypeptide chain comprises a signal sequence; an anti-ALFA Dab followed by a CD8 stalk; a transmembrane domain, a linker; a second wobbled anti-ALFA Dab; a truncated CD20 (containing its N-terminus, TM and small loop) followed by the KDEL sequence on the C-terminus (see FIG. 5). The construct is transduced into PD1 positive Jurkats and activated PBMCs and the surface expression level of HLA is assessed by flow cytometry. The functional consequence of sequestering SHP2 is assessed through killing, cytokine secretion and proliferative responses to co-cultures with target cells expressing cognate ligand (CD19) and PDL1.

### Example 5—Proof of Concept Experiment for a “Peptide-Tag” Linked Binder

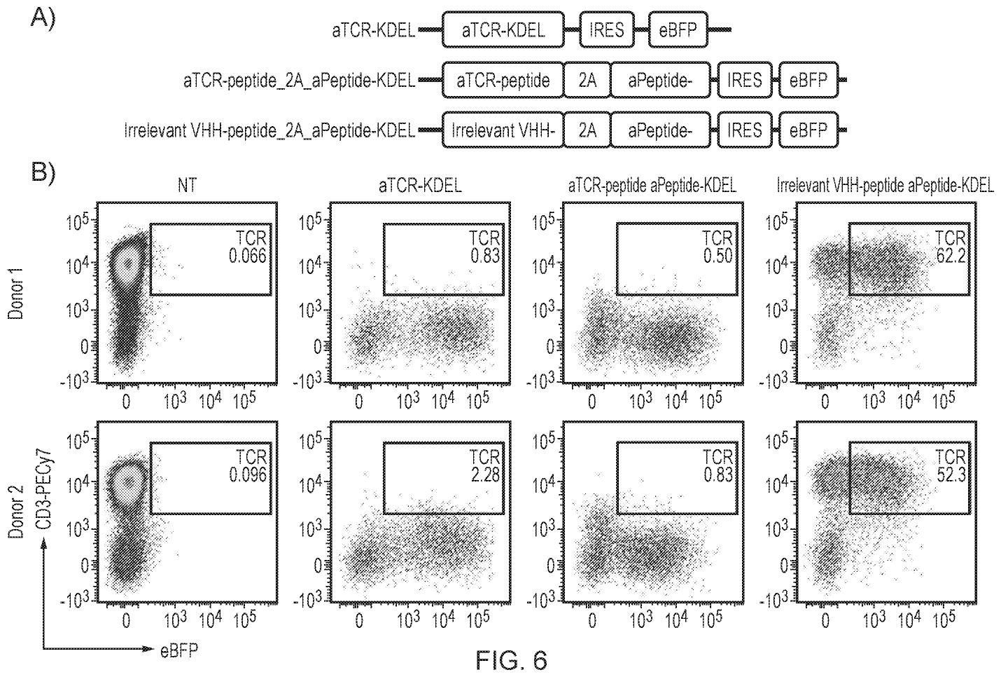

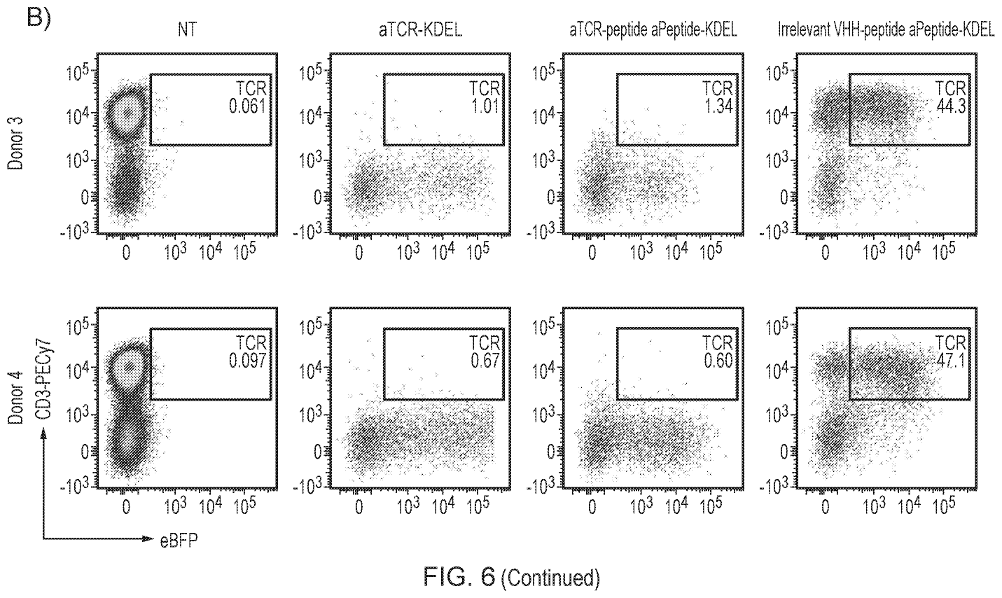

In order to demonstrate that KDEL driven TCR knock-down can be mediated through a dual polypeptide chain construct, PBMC's were transduced to express either a single polypeptide encoding anti-TCR_VHH directly linked to a KDEL sequence; or two polypeptide chains: the first encoding an anti-TCR_VHH linked to an ALFA_peptide; and the second encoding an anti-ALFA_peptide_VHH directly linked to a KDEL sequence (FIG. 6A). The two polypeptides were separated by a self-cleaving 2A peptide. As a negative control the aTCR_VHH was substituted with an irrelevant VHH binder. All constructs contained an IRES-eBFP marker for transduction.

Four days following transduction, PBMCs were stained for surface CD3 and analysed by flow cytometry. The results are from four independent donors are shown in FIG. 6B. Expression of TCR at the cell surface was dramatically reduced in cells expressing either the anti-TCR-KDEL or in cells expressing the two polypeptide chains: an anti-TCR VHH-peptide; and anti-peptide VHH-KDEL. No significant reduction in cell-surface TCR expression was seen in cells expressing the two polypeptide chains: the irrelevant VHH-peptide; and anti-peptide VHH-KDEL. This demonstrates that a peptide tag-linked binder can be used with a anti-peptide KDEL to block cell surface expression of a target protein such as TCR. A similar approach could be used to block or reduce surface expression of two or more proteins, by using peptide tag-linked binders with different target-binding domains, but the same peptide. Both or all of such peptide tag-linked binders (i.e. target-binding polypeptides) are retained inside the intracellular compartment by the same anti-peptide-binding KDEL (i.e. the same localizing polypeptide)

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1-23. (canceled)

24. An engineered immune cell comprising one or more nucleic acid constructs which together encode at least two target-binding domains, wherein the one or more nucleic acid constructs together contain a single nucleotide sequence encoding an intracellular retention signal which, when co-expressed in the cell, controls the cellular localization of each of the target-binding domains.

25. An engineered immune cell according to claim 24, which comprises a nucleic acid construct encoding an engineered protein which comprises at least two target-binding domains coupled to an intracellular retention signal.

26. An engineered immune cell according to claim 24, which comprises
(i) at least two target-binding polypeptides, each of which comprise a target-binding domain and a first protein interaction domain, and
(ii) a localizing polypeptide comprising a second protein interaction domain, which binds to the first protein binding domain of each of the target binding polypeptides, and an intracellular retention signal.

27. A nucleic acid construct which comprises the following structure:
A-X-B-C
in which:
A and B are nucleic acid sequences each encoding a target-binding domain, X is a nucleic acid sequence encoding a linker, and C is nucleic acid sequence encoding an intracellular retention signal.

28. A nucleic acid construct which comprises:
(i) a plurality of nucleic acid sequences each of which encodes a target binding polypeptide comprising a target-binding domain and a first protein interaction domain, and
(ii) a nucleic acid sequence which encodes a localizing polypeptide comprising a second protein interaction domain, which binds to the first protein binding domain, and an intracellular retention signal.

29. (canceled)

30. A vector which comprises a nucleic acid construct according to claim 27.

31. A vector which comprises a nucleic acid construct according to claim 28.

32. (canceled)

33. A method for making an engineered immune cell, which comprises the step of introducing into an immune cell a vector according to claim 30 into a cell.

34. A method for making an engineered immune cell, which comprises the step of introducing into an immune cell a vector according to claim 31 into a cell.

35. A pharmaceutical composition which comprises an engineered immune cell according to claim 24.

36. A method for treating a disease, which comprises the step of administering a pharmaceutical composition according to claim 35 to a subject in need thereof.

37. The method according to claim 36, wherein the cell is autologous.

38. The method according to claim 36, wherein the cell is allogenic.


SEQUENCE LISTING (180 sequences)

SEQ ID NO: 1 - protein, 6 aa, Artificial Sequence
  motif to direct proteins to the Golgi apparatus
SEKDEL

SEQ ID NO: 2 - protein, 4 aa, Artificial Sequence
  Golgi retention signal sequence
KDEL

SEQ ID NO: 3 - protein, 4 aa, Artificial Sequence
  Golgi retention signal sequence
  misc_feature (3)..(4): Xaa can be any naturally occurring amino acid
KKXX

SEQ ID NO: 4 - protein, 5 aa, Artificial Sequence
  Golgi retention signal sequence
  misc_feature (2)..(2): Xaa can be any naturally occurring amino acid
  misc_feature (4)..(5): Xaa can be any naturally occurring amino acid
KXKXX

SEQ ID NO: 5 - protein, 15 aa, Artificial Sequence
  Golgi retention signal sequence
KYKSRRSFIDEKKMP

SEQ ID NO: 6 - protein, 10 aa, Artificial Sequence
  Golgi retention signal sequence
MHRRRSRSCR

SEQ ID NO: 7 - protein, 3 aa, Artificial Sequence
  Golgi retention signal sequence
  misc_feature (2)..(2): Xaa can be any naturally occurring amino acid
  MISC_FEATURE (3)..(3): Xaa may be Asp or Glu
KXX

SEQ ID NO: 8 - protein, 4 aa, Artificial Sequence
  Golgi retention signal sequence
YQRL

SEQ ID NO: 9 - protein, 5 aa, Artificial Sequence
  linker
GGGGS

SEQ ID NO: 10 - protein, 10 aa, Artificial Sequence
  linker
GGGGSGGGGS

SEQ ID NO: 11 - protein, 13 aa, Artificial Sequence
  linker
SGGGSGGGSGGGS

SEQ ID NO: 12 - protein, 111 aa, Artificial Sequence
  ectodomain from CD79a
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNR

SEQ ID NO: 13 - protein, 131 aa, Artificial Sequence
  ectodomain from CD79b
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 14 - protein, 107 aa, Artificial Sequence [sequence omitted]

SEQ ID NO: 15 - protein, 97 aa, Artificial Sequence
  CH1 region
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 16 - protein, 24 aa, Artificial Sequence
  transmembrane domain, Tyrp-TM
IIAIAVVGALLLVALIFGTASYLI

SEQ ID NO: 17 - protein, 22 aa, Artificial Sequence
  transmembrane domain, dCD20_TM
IMNGLFHIALGGLLMIPAGIYA

SEQ ID NO: 18 - protein, 47 aa, Artificial Sequence
  spacer domain, CD8STK
PTTTPAPRPPTPAPTIASQPLSLRPEACRPAAGGAVHTRGLDFACDI

SEQ ID NO: 19 - protein, 55 aa, Artificial Sequence
  spacer domain, dCD20_N-terminal
TTPRNSVNGTFPAEPMKGPIAMQSGPKPLFRRMSSLVGPTQSFFMRESKTLGAVQ

SEQ ID NO: 20 - protein, 6 aa, Artificial Sequence
  spacer domain, dCD20_Short_Loop
PICVTV

SEQ ID NO: 21 - protein, 15 aa, Artificial Sequence
  ALFA_tag peptide
PSRLEEELRRRLTEP

SEQ ID NO: 22 - protein, 122 aa, Artificial Sequence
  anti-ALFA_Tag (nanobody NbALFA) peptide
EVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 23 - protein, 9 aa, Artificial Sequence
  epitope tag system, HA-tag
YPYDVPDYA

SEQ ID NO: 24 - protein, 8 aa, Artificial Sequence
  epitope tag system, FLAG-tag
DYKDDDDK

SEQ ID NO: 25 - protein, 12 aa, Artificial Sequence
  epitope tag system, SPOT-tag
PDRVRAVSHWSS

SEQ ID NO: 26 - protein, 4 aa, Artificial Sequence
  epitope tag system, EPEA/C-tag
EPEA

SEQ ID NO: 27 - protein, 10 aa, Artificial Sequence
  epitope tag system, myc-tag
EQKLISEEDL

SEQ ID NO: 28 - protein, 6 aa, Artificial Sequence
  last 6 amino acids of a tail of adenoviral E19 protein
DEKKMP

SEQ ID NO: 29 - protein, 7 aa, Artificial Sequence
  intracellular retention signal, sequence from TYRP-1
NQPLLTD

SEQ ID NO: 30 - protein, 245 aa, Artificial Sequence
  target binding domain, Ab-1 (aCD3e_UCHT)
DIQMTQSPSSLSASVGNRVTITCRASQDIRNYLNWYQQKPGKAPKLLIYYTSRLESGVPSRFSGSGSGTDYTLTISSLQPEDFATYYCQQGNTLPWTFGQGTKVEIKSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 31 - protein, 128 aa, Artificial Sequence
  target binding domain, Ab-2 (aB2M_dN6B2m)
QVQLQESGGGSVQAGGSLRLSCAASGYTDSRYCMAWFRQAPGKEREWVARINSGRDITYYADSVKGRFTFSQDNAKNTVYLQMDSLEPEDTATYYCATDIPLRCRDIVAKGGDGFRYWGQGTQVTVSS

SEQ ID NO: 32 - protein, 248 aa, Artificial Sequence
  target binding domain, Ab-3 (aPD1_clone10)
DVLMTQTPLSLPVSLGDQASISCRSGQNIVHSNGNTYLEWYLQKPGQSPKLLIYKVSNRFFGVPDRISGSGSGTDFTLKISRVEAEDLGVYFCFQGSHVPFTFGSGTKLEIKSGGGGSGGGGSGGGGSDVQLQESGPGLVKPSQSLSLTCTVTGYSITSDYAWNWIRQFPGNKLEWMGYINYSGSTSYNPSLKSRISITRDTSKNQFFLQLNSVTTEDTATYYCARWIGSSAWYFDVWGAGTTVTVSS

SEQ ID NO: 33 - protein, 474 aa, Artificial Sequence
  chimeric antigen receptor (CAR) sequence, aCD19CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 138 aa, Artificial Sequence
  marker, RQR8
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRA

SEQ ID NO: 35 - protein, 20 aa, Artificial Sequence
  signal peptide, murine Ig kappa chain V-III region
METDTLILWVLLLLVPGSTG

SEQ ID NO: 36 - protein, 21 aa, Artificial Sequence
  signal peptide
MGTSLLCWMALCLLGADHADA

SEQ ID NO: 37 - protein, 18 aa, Artificial Sequence
  self-cleaving peptide, 2A-like sequence
EGRGSLLTCGDVEENPGP

SEQ ID NO: 38 - protein, 474 aa, Artificial Sequence; identical to SEQ ID NO: 33
  CAR sequence, aCD19CAR

SEQ ID NO: 39 - protein, 244 aa, Artificial Sequence
  target binding domain, Ab-4 (aCD52_)
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKSGGGGSGGGGSGGGGSQVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSS

SEQ ID NO: 40 - protein, 112 aa, Artificial Sequence
  target binding domain, Ab-5 (aTBR2_E11)
QVQLQESGGGLVQPGGSLRLSCAASGIILSSKAVAWYRQPPGQQREGVAHSSVSGTTIYADSVKGRFTVSRDNAKNTVYLEMNSLKPEDTAVYYCTAPVGHWGQGTQVTVSS

SEQ ID NO: 41 - protein, 5 aa, Artificial Sequence
  intracellular retention signal sequence
  misc_feature (4)..(4): Xaa can be any naturally occurring amino acid
NPFXD

SEQ ID NO: 42 - protein, 4 aa, Artificial Sequence
  tyrosine-based sorting signal consensus motif
  misc_feature (3)..(3): Xaa can be any naturally occurring amino acid
NPXY

SEQ ID NO: 43 - protein, 4 aa, Artificial Sequence
  tyrosine-based sorting signal consensus motif
  misc_feature (2)..(3): Xaa can be any naturally occurring amino acid
  MISC_FEATURE (4)..(4): Xaa is an amino acid with a bulky hydrophobic side chain
YXXX

SEQ ID NO: 44 - protein, 12 aa, Homo sapiens
INFDNPVYQKTT

SEQ ID NO: 45 - protein, 12 aa, Homo sapiens
VEIGNPTYKMYE

SEQ ID NO: 46 - protein, 12 aa, Homo sapiens
TNFTNPVYATLY

SEQ ID NO: 47 - protein, 12 aa, Drosophila sp.
GNFANPVYESMY

SEQ ID NO: 48 - protein, 12 aa, Caenorhabditis elegans
TTFTNPVYELED

SEQ ID NO: 49 - protein, 12 aa, Caenorhabditis elegans
LRVDNPLYDPDS

SEQ ID NO: 50 - protein, 12 aa, Homo sapiens
IIFENPMYSARD

SEQ ID NO: 51 - protein, 12 aa, Homo sapiens
TNFENPIYAQME

SEQ ID NO: 52 - protein, 12 aa, Homo sapiens
DTGENPIYKSAV

SEQ ID NO: 53 - protein, 11 aa, Homo sapiens
TTVVNPKYEGK

SEQ ID NO: 54 - protein, 12 aa, Drosophila sp.
WDTENPIYKQAT

SEQ ID NO: 55 - protein, 11 aa, Drosophila sp.
STFKNPMYAGK

SEQ ID NO: 56 - protein, 12 aa, Homo sapiens
HGYENPTYRFLE

SEQ ID NO: 57 - protein, 12 aa, Homo sapiens
NGYENPTYKFFE

SEQ ID NO: 58 - protein, 12 aa, Drosophila sp.
NGYENPTYKYFE

SEQ ID NO: 59 - protein, 12 aa, Homo sapiens
YASSNPEYLSAS

SEQ ID NO: 60 - protein, 12 aa, Homo sapiens
GSVQNPVYHNQP

SEQ ID NO: 61 - protein, 12 aa, Homo sapiens
TAVGNPEYLNTV

SEQ ID NO: 62 - protein, 12 aa, Homo sapiens
ISLDNPDYQQDF

SEQ ID NO: 63 - protein, 11 aa, Homo sapiens
RKRSHAGYQTI

SEQ ID NO: 64 - protein, 10 aa, Homo sapiens
KHHHAGYEQF

SEQ ID NO: 65 - protein, 11 aa, Gallus gallus
KKHHNTGYEQF

SEQ ID NO: 66 - protein, 11 aa, Gallus gallus
RRKSRTGYQSV

SEQ ID NO: 67 - protein, 11 aa, Gallus gallus
RRKSYAGYQTL

SEQ ID NO: 68 - protein, 12 aa, Drosophila sp.
RRRSTSRGYMSF

SEQ ID NO: 69 - protein, 11 aa, Lumbricus rubellus
RKRSRRGYESV

SEQ ID NO: 70 - protein, 10 aa, Homo sapiens
KSIRSGYEVM

SEQ ID NO: 71 - protein, 12 aa, Homo sapiens
HCGGPRPGYETL

SEQ ID NO: 72 - protein, 12 aa, Mus musculus
HCRTRRAEYETL

SEQ ID NO: 73 - protein, 10 aa, Homo sapiens
RRRPSAYQAL

SEQ ID NO: 74 - protein, 9 aa, Homo sapiens
RRRSYQNIP

SEQ ID NO: 75 - protein, 10 aa, Homo sapiens
KKHCSYQDIL

SEQ ID NO: 76 - protein, 10 aa, Mus musculus
RRRSAYQDIR

SEQ ID NO: 77 - protein, 11 aa, Rattus norvegicus
RKRRRSYQDIM

SEQ ID NO: 78 - protein, 13 aa, Rattus norvegicus
KFCKSKERNYHTL

SEQ ID NO: 79 - protein, 14 aa, Drosophila sp.
KFYKARNERNYHTL

SEQ ID NO: 80 - protein, 14 aa, Homo sapiens
KIRLRCQSSGYQRI

SEQ ID NO: 81 - protein, 14 aa, Mus musculus
KIRQRHQSSAYQRI

SEQ ID NO: 82 - protein, 15 aa, Homo sapiens
HFCLYRKRPGYDQLN

SEQ ID NO: 83 - protein, 11 aa, Homo sapiens
SLSRGSGYKEI

SEQ ID NO: 84 - protein, 13 aa, Homo sapiens
RRLRKGYTPLMET

SEQ ID NO: 85 - protein, 14 aa, Homo sapiens
RRAGHSSYTPLPGS

SEQ ID NO: 86 - protein, 18 aa, Dictyostelium discoideum
KKLRQQKQQGYQAIINNE

SEQ ID NO: 87 - protein, 15 aa, Dictyostelium discoideum
RSKSNQNQSYNLIQL

SEQ ID NO: 88 - protein, 17 aa, Dictyostelium discoideum
RKTFYNNNQYNGYNIIN

SEQ ID NO: 89 - protein, 10 aa, Homo sapiens
PLSYTRFSLA

SEQ ID NO: 90 - protein, 11 aa, Homo sapiens
MTKEYQDLQHL

SEQ ID NO: 91 - protein, 10 aa, Homo sapiens
SYKYSKVNKE

SEQ ID NO: 92 - protein, 10 aa, Homo sapiens
PAAYRGVGDD

SEQ ID NO: 93 - protein, 10 aa, Homo sapiens
TGVYVKMPPT

SEQ ID NO: 94 - protein, 10 aa, Homo sapiens
LISYKGLPPE

SEQ ID NO: 95 - protein, 11 aa, Rattus norvegicus
ASDYQRLNLKL

SEQ ID NO: 96 - protein, 11 aa, Human immunodeficiency virus type 1
RQGYSPLSFQT

SEQ ID NO: 97 - protein, 19 aa, Homo sapiens
RMQAQPPGYRHVADGEDHA

SEQ ID NO: 98 - protein, 7 aa, Artificial Sequence
  dileucine-based sorting signal sequence
  MISC_FEATURE (1)..(1): Xaa may be Asp or Glu
  misc_feature (2)..(4): Xaa can be any naturally occurring amino acid
  MISC_FEATURE (7)..(7): Xaa may be Leu or Ile
XXXXLLX

SEQ ID NO: 99 - protein, 9 aa, Homo sapiens
SDKQTLLPN

SEQ ID NO: 100 - protein, 9 aa, Rattus norvegicus
DERAPLIRT

SEQ ID NO: 101 - protein, 9 aa, Homo sapiens
QEKDPLLKN

SEQ ID NO: 102 - protein, 9 aa, Odontophorus gujanensis
TERNPLLKS

SEQ ID NO: 103 - protein, 9 aa, Homo sapiens
GENSPLLSG

SEQ ID NO: 104 - protein, 9 aa, Homo sapiens
EEKQPLLME

SEQ ID NO: 105 - protein, 9 aa, Oryzias latipes
GERQPLLQS

SEQ ID NO: 106 - protein, 9 aa, Gallus gallus
PEIQPLLTE

SEQ ID NO: 107 - protein, 9 aa, Carassius auratus
EGRQPLLGD

SEQ ID NO: 108 - protein, 9 aa, Homo sapiens
EANQPLLTD

SEQ ID NO: 109 - protein, 9 aa, Gallus gallus
ELHQPLLTD

SEQ ID NO: 110 - protein, 9 aa, Danio rerio
REFEPLLNA

SEQ ID NO: 111 - protein, 9 aa, Homo sapiens
EEKMAILMD

SEQ ID NO: 112 - protein, 9 aa, Homo sapiens
EEKLAILSQ

SEQ ID NO: 113 - protein, 9 aa, Mus musculus
SERDVLLDE

SEQ ID NO: 114 - protein, 9 aa, Homo sapiens
SERRNLLED

SEQ ID NO: 115 - protein, 9 aa, Rattus norvegicus
DDSGDLLPG

SEQ ID NO: 116 - protein, 9 aa, Homo sapiens
SQIKRLLSE

SEQ ID NO: 117 - protein, 9 aa, Felis catus
SHIKRLLSE

SEQ ID NO: 118 - protein, 9 aa, Mus musculus
RRTPSLLEQ

SEQ ID NO: 119 - protein, 9 aa, Homo sapiens
HRTPSLLEQ

SEQ ID NO: 120 - protein, 10 aa, Rattus norvegicus
EPRGSRLLVR

SEQ ID NO: 121 - protein, 19 aa, Homo sapiens
MDDQRDLISNNEQLPMLGR

SEQ ID NO: 122 - protein, 19 aa, Mus musculus
MDDQRDLISNHEQLPILGN

SEQ ID NO: 123 - protein, 19 aa, Gallus gallus
MDDQRDLISNHEQLPILGN

SEQ ID NO: 124 - protein, 23 aa, Danio rerio
MEPDHQNESLIQRVPSAETILGR

SEQ ID NO: 125 - protein, 23 aa, Danio rerio
MSSEGNETPLISDQSSVNMGPQP

SEQ ID NO: 126 - protein, 24 aa, Trypanosoma sp.
RPRRRTEEDELLPEEAEGLIDPQN

SEQ ID NO: 127 - protein, 19 aa, Homo sapiens
PDKHSLLVGDFREDDDTAL

SEQ ID NO: 128 - protein, 9 aa, Homo sapiens
TERERLLNF

SEQ ID NO: 129 - protein, 8 aa, Homo sapiens
VETDDLIL

SEQ ID NO: 130 - protein, 7 aa, Caenorhabditis elegans
FENDSLL

SEQ ID NO: 131 - protein, 9 aa, Saccharomyces cerevisiae
NEQSPLLHN

SEQ ID NO: 132 - protein, 8 aa, Saccharomyces cerevisiae
SEQTRLVP

SEQ ID NO: 133 - protein, 8 aa, Saccharomyces cerevisiae
EVDLDLLK

SEQ ID NO: 134 - protein, 5 aa, Artificial Sequence
  dileucine-based sorting signal sequence
  misc_feature (2)..(3): Xaa can be any naturally occurring amino acid
DXXLL

SEQ ID NO: 135 - protein, 13 aa, Homo sapiens
SFHDDSDEDLLHI

SEQ ID NO: 136 - protein, 13 aa, Bos taurus
TFHDDSDEDLLHV

SEQ ID NO: 137 - protein, 13 aa, Oryctolagus cuniculus
SFHDDSDEDLLNI

SEQ ID NO: 138 - protein, 13 aa, Gallus gallus
SFHDDSDEDLLNV

SEQ ID NO: 139 - protein, 13 aa, Homo sapiens
EESEERDDHLLPM

SEQ ID NO: 140 - protein, 13 aa, Gallus gallus
DESEERDDHLLPM

SEQ ID NO: 141 - protein, 12 aa, Homo sapiens
GYHDDSDEDLLE

SEQ ID NO: 142 - protein, 13 aa, Homo sapiens
ITGFSDDVPMVIA

SEQ ID NO: 143 - protein, 13 aa, Hydra sp.
INRFSDDEPLVVA

SEQ ID NO: 144 - protein, 13 aa, Homo sapiens
MLEASDDEALLVC

SEQ ID NO: 145 - protein, 13 aa, Homo sapiens
KNETSDDEALLLC

SEQ ID NO: 146 - protein, 12 aa, Mus musculus
WVVEAEDEPLLA

SEQ ID NO: 147 - protein, 12 aa, Homo sapiens
WVAEAEDEPLLT

SEQ ID NO: 148 - protein, 12 aa, Homo sapiens
HDDFADDISLLK

SEQ ID NO: 149 - protein, 13 aa, Mus musculus
GRDSPEDHSLLVN

SEQ ID NO: 150 - protein, 12 aa, Peromyscus maniculatus
VRCHPEDDRLLG

SEQ ID NO: 151 - protein, 13 aa, Homo sapiens
HRVSQDDLDLLTS

SEQ ID NO: 152 - protein, 13 aa, Homo sapiens
ASVSLLDDELMSL

SEQ ID NO: 153 - protein, 13 aa, Homo sapiens
ASSGLDDLDLLGK

SEQ ID NO: 154 - protein, 13 aa, Homo sapiens
VQNPSADRNLLDL

SEQ ID NO: 155 - protein, 13 aa, Homo sapiens
NALSWLDEELLCL

SEQ ID NO: 156 - protein, 13 aa, Drosophila sp.
TVDSIDDVPLLSD

SEQ ID NO: 157 - protein, 13 aa, Mus musculus
QEECPSDSEEDEG

SEQ ID NO: 158 - protein, 13 aa, Mus musculus
RDRDYDEDDEDDI

SEQ ID NO: 159 - protein, 15 aa, Mus musculus
LDETEDDELEYDDES

SEQ ID NO: 160 - protein, 10 aa, Homo sapiens
KDPDEVETES

SEQ ID NO: 161 - protein, 13 aa, Homo sapiens
HEFQDETDTEEET

SEQ ID NO: 162 - protein, 14 aa, Homo sapiens
QEKEDDGSESEEEY

SEQ ID NO: 163 - protein, 9 aa, Homo sapiens
GEDEESESD

SEQ ID NO: 164 - protein, 12 aa, Homo sapiens
GEDSDEEPDHEE

SEQ ID NO: 165 - protein, 11 aa, Homo sapiens
LEDDSDEEEDF

SEQ ID NO: 166 - protein, 9 aa, Human cytomegalovirus
KDSDEEENV

SEQ ID NO: 167 - protein, 13 aa, Herpes simplex virus 3
FEDSESTDTEEEF

SEQ ID NO: 168 - protein, 9 aa, Human immunodeficiency virus type 1
LEAQEEEEV

SEQ ID NO: 169 - protein, 22 aa, Saccharomyces cerevisiae
ADDLESGLGAEDDLEQDEQLEG

SEQ ID NO: 170 - protein, 12 aa, Saccharomyces cerevisiae
TEIDESFEMTDF

SEQ ID NO: 171 - protein, 18 aa, Saccharomyces cerevisiae
TEPEEVEDFDFDLSDEDH

SEQ ID NO: 172 - protein, 15 aa, Saccharomyces cerevisiae
FEIEEDDVPTLEEEH

SEQ ID NO: 173 - protein, 3 aa, Artificial Sequence
  intracellular retention signal sequence
  MISC_FEATURE (3)..(3): Xaa may be Phe or Trp
DPX

SEQ ID NO: 174 - protein, 5 aa, Artificial Sequence
  intracellular retention signal sequence
  misc_feature (2)..(2): Xaa can be any naturally occurring amino acid
  misc_feature (4)..(4): Xaa can be any naturally occurring amino acid
FXDXF

SEQ ID NO: 175 - protein, 5 aa, Artificial Sequence
  intracellular retention signal sequence
  MISC_FEATURE (2)..(2): Xaa is an amino acid with a bulky hydrophobic side chain
  misc_feature (3)..(3): Xaa can be any naturally occurring amino acid
  MISC_FEATURE (4)..(4): Xaa is an amino acid with a bulky hydrophobic side chain
  MISC_FEATURE (5)..(5): Xaa may be Asp or Glu
LXXXX

SEQ ID NO: 176 - protein, 5 aa, Artificial Sequence
  intracellular retention signal sequence
LLDLL

SEQ ID NO: 177 - protein, 5 aa, Artificial Sequence
  intracellular retention signal sequence
PWDLW

SEQ ID NO: 178 - protein, 4 aa, Artificial Sequence
  intracellular retention signal sequence
HDEL

SEQ ID NO: 179 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

SEQ ID NO: 180 - protein, 7 aa, Artificial Sequence
  consensus Tobacco Etch Virus (TEV) cleavage site
ENLYFQS